(19) United States
(12) Patent Application Publication
(10) Pub. No.: US 2011/0079733 A1
(43) Pub. Date: Apr. 7, 2011

(54) FLUORESCENT, HETEROCYCLICALLY FUSED PERYLENES

(75) Inventors: Heinz Langhals, Ottobrunn (DE); Simon Kinzel, Munchen (DE); Andreas Obermeier, Haimhausen (DE)

(73) Assignee: BASF SE, Tarrytown, NY (US)

(21) Appl. No.: 12/745,900

(22) PCT Filed: Dec. 12, 2008
(86) PCT No.: PCT/EP08/67416

§ 371 Date: Dec. 7, 2010

(30) Foreign Application Priority Data
Dec. 12, 2007 (DE) 102007059683.0
Dec. 10, 2008 (DE) 102008061452.1

(51) Int. Cl. G01N 21/64; C07D 471/16; C07D 471/22

(52) U.S. Cl. 250/461.1; 546/32; 546/28; 546/27; 540/555; 250/200

(57) ABSTRACT

The reaction of aromatic nitriles with perylenetetracarboximides affords colorants which fluoresce strongly in the long-wave region and are of the formula

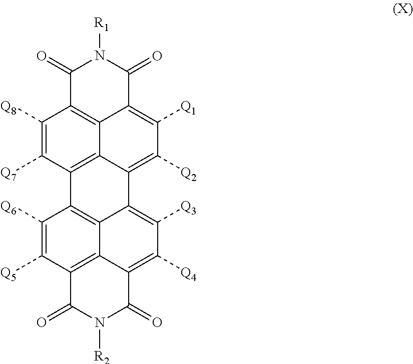

in which one pair Q₁ and Q₂ or one pair Q₂ and Q₃, and optionally from 0 to 3 pairs Q₃ and Q₄, Q₅ and Q₆, Q₆ and Q₇ and/or Q₇ and Q₈, in each case in pairs, together form a heterocyclic ring

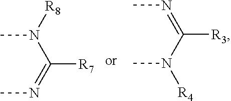

the remaining Q₁, Q₄, Q₅ and Q₈ which do not form a heterocyclic ring are each hydrogen, and the remaining Q₃, Q₆ and Q₇ which do not form a heterocyclic ring are each independently R₅ or R₆.

R₁ to R₈ are each H, CN, halogen or optionally mono- or polysubstituted hydrocarbon groups.

Establishing a suitable pH achieves a large Stokes shift in these colorants via the ESPT mechanism.

As a result of the increasing scarcity of fossil energy carriers and the carbon dioxide emission thereof, which is leading to global warming via the greenhouse effect, there is increasing industrial interest in alternative energy sources, the utilization of which does not have an adverse effect on the environment. Among these, solar energy is particularly attractive, because it appears to be unlimited on the human scale. The main problem is, however, the low energy density of this source, which is extremely serious for industrial utilization. The problem can in principle be solved by light-concentrating systems. If the intention is also to utilize the diffuse solar radiation which is important in the temperate latitudes, systems with nonlinear optics are required. Particular mention should be made here of the fluorescent solar collector [Nachr. Chem. Tech. Lab. 1980, 28, 716-718], which consists of a plane-parallel plate of high-refractive material colored with a fluorescent dye. For the short-wave visible region, there are some available colorants, for example the perylene colorants (1), which fluoresce with high quantum yields. In contrast, the long-wave visible and NIR region constitutes the greatest problem.

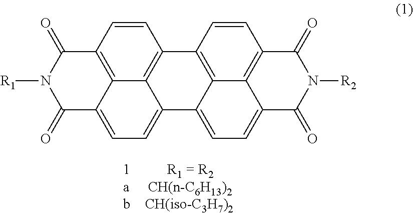

It was an object of the invention to develop a broadband-absorbing fluorescent colorant which fluoresces in the long-wave visible range in order to be able to use it for applications such as light-collecting systems, for example in fluorescent solar collectors.

The lightfastness of the perylenetetracarboximides (1) [Helv. Chim. Acta. 2005, 88, 1309-1343; Heterocycles 1995, 40, 477-500] and the high fluorescence quantum yields thereof mean that the compound class is of exceptional interest for solar applications, for example in the fluorescent solar collector. With λₘₐₓ of approx. 525 nm, however, only the short-wave visible region of solar radiation can be utilized by (1). There is a whole series of approaches for shifting the light absorption of the perylenebisimides to the longer-range region. For example, the exchange of the carbonyl groups in (1) for ketimino groups has led to a significant bathochromic shift in the absorption [J. Prakt. Chem. 1997, 339, 597-602; Liebigs Ann. Chem. 1995, 481-486; Chem. Ber. 1983, 116, 3524-3528]. According to Adachi [Pure Appl. Chem. 1996, 68, 1441-1442], however, the bathochromic shift achievable by this concept is limited.

do.-π-Ac.-π-do. (a)

ac.-π-Do.-π-ac. (b)

As an alternative, for a long-wave shift, the perylene ring system has been substituted by donor groups in positions 1, 6, 7 and 12 [Forschungsber.-Bundesminist. Forsch. Technol., Technol. Forsch. Entwickl. 1984, BMFT-FB-T 84-164; Chem. Abstr. 1985, 102, 150903; Vestn. Khar'kov. Politekh. Inst. 1969, 41, 21-26; Chem. Abstr. 1971, 75, 7375; Tetrahedron Lett. 1999, 40, 7047-7050; Eur. J. Org. Chem. 2000, 365-380] (these positions are frequently referred to in the literature as the ‘bay region’—this expression is not used here since it gives the incorrect impression that a sterically particularly readily obtainable structure is present here; at these positions, however, the steric pressure is particularly great because there is an accumulation of hydrogen atoms which is not expressed in the customary line notation of the formulae).

The chromophore of the perylenebisimides can be interpreted as an inverse Konig dye system; in the normal dye system according to equation (a) [Prakt. Chem. 1926, 112, 1-36], two terminal donor groups are joined via π systems to a central acceptor. In the case of inverse systems, according to equation (b), donor and acceptor groups are switched. In the perylenebisimides, the carbonyl groups constitute these terminal acceptor groups and the donor groups in the center are absent [Helv. Chim. Acta. 2005, 88, 1309-1343]. Accordingly, donor groups in these positions bring about a bathochromic shift in the absorption, which, in the case of use of α-effect donor groups [Dyes Pigm. 2003, 59, 109-116], extends into the NIR region. To date, only five- and six-membered rings have been fused onto these positions. This raises the question of to what extent other ring sizes lead to colorants with long-wave-absorbing colorants of interest.

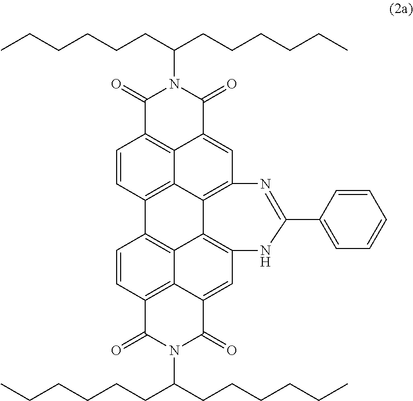

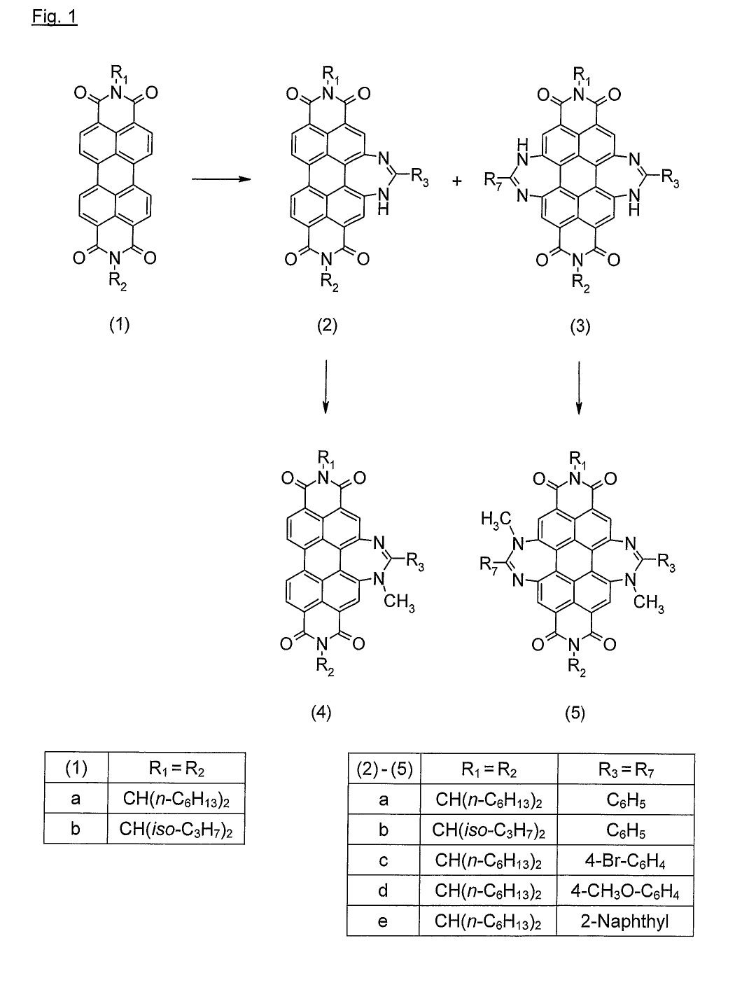

We allowed a mixture of sodium amide and benzonitrile to act on the colorant (1a) [Chem. Ber. 1988, 121, 225-230] under atmospheric oxygen at a temperature of 160° C., and astonishingly directly achieved a ring closure to give a seven-membered ring with formation of (2a), in which a seven-membered ring with two nitrogen atoms is fused onto (1a); see FIG. 1. In the case of an excess of the reagent mixture, the reaction also proceeds twice to form the substances (3a). This reaction is not restricted solely to the substrate (1a) and benzonitrile, but is surprisingly universal. For instance, the reaction has also been verified using the example of the more complicated starting material (1b) [Angew. Chem. 2006, 118, 4555-4561; Angew. Chem. Int. Ed. Engl. 2006, 45, 4444-4447] to form (2b) and (3b). Various other aromatic nitriles have been converted, for example 4-bromobenzonitrile to (2c) and (3c), 4-methoxybenzonitrile to (2d) and (3d), and it has even been possible to convert nitriles of polycyclic aromatics, as shown by the reaction of 2-naphthonitrile to give (2e) and (3e). The reaction can be performed in substance or in solution. However, the solvent must be sufficiently stable under the strongly alkaline reaction conditions, an example being 1,2-dimethoxyethane. The nitriles can surprisingly be replaced by the carboxamides, for example benzamide—this does not significantly adversely affect the yields of (2) and (3). The strong sodium amide base can be replaced by solid potassium hydroxide; sodium hydride affords significantly poorer results. For the progress of the reaction, the ingress of atmospheric oxygen is required, since no conversion whatsoever was detected under a protective argon atmosphere.

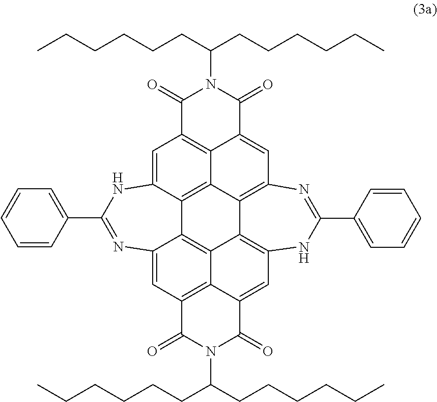

The substances (2) and (3) each comprise acidic protons on one or two nitrogen atoms. This raises the question of whether these positions can be converted after a deprotonation with electrophiles. Although such anions are also present spontaneously in the protonation equilibria, we expected more favorable results after a deprotonation using strong bases. We deprotonated each of substances (2) and (3) with sodium hydride as a typical strong base and then reacted them with methyl iodide as an electrophile. A reaction under these conditions proceeded astonishingly smoothly with formation of the methyl derivatives.

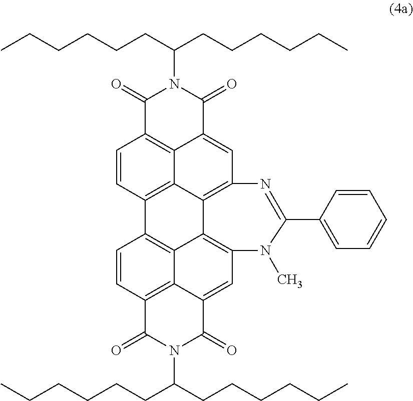

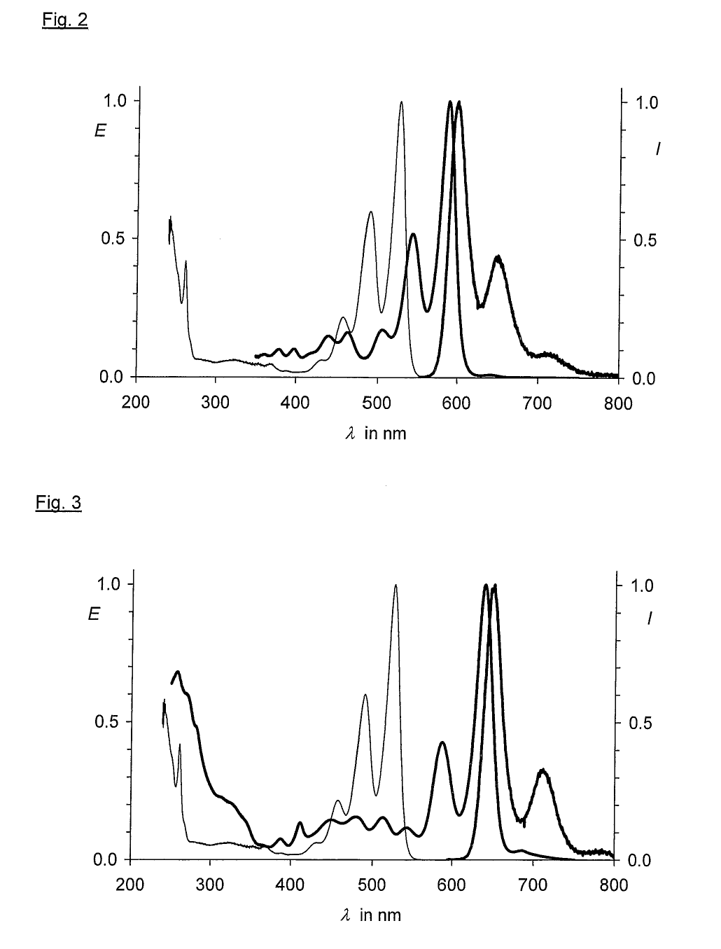

The UV/Vis spectroscopy properties of the novel colorant classes are just as surprising as the simple synthesis thereof. The UV/Vis absorption of (2) is shown in FIG. 2—compared to (1a), a considerable bathochromic color shift is observed. Even more astonishing is the marked red fluorescence of the substance, the quantum yield of which is close to 100%. This is remarkable in that the literature speculates that high fluorescence quantum yields are not possible in the long-wave visible spectral region owing to coupling with C—H vibration overtones. The fluorescence of (2) surprisingly disproves this postulation. The substance is outstandingly lightfast and is also suitable for applications under the direct action of sunlight. The novel substance (2) has the advantage over the abovementioned donor-substituted perylene derivatives that there is not only a bathochromic shift in the longest-wave absorption but, in addition, there are additional further bands in the visible and UV region, which are attributable to the new heterocyclic structure. These new bands have the effect that light is absorbed by the longest-wave absorption into the UV region, such that the absorber is a broadband absorber. Such broadband absorbers are of particular interest for any kind of solar applications, especially for light-collecting systems, which can thus utilize a correspondingly large section from solar radiation.

In the novel heterocyclic colorant (3) with two seven-membered rings, an even stronger bathochromic shift in the absorption occurs, such that even green solutions are obtained; see FIG. 3. The absorber here is an extreme broadband absorber which can absorb light continuously from the UV into the NIR region. This colorant too fluoresces with a quantum yield close to 100%. Since the predominant portion of the fluorescence occurs in the long-wave visible region with an already falling eye sensitivity and large parts of the spectrum are in the NIR, the deep red fluorescence no longer appears to be as marked as that of (2).

Strong bases such as DBU can be used to deprotonate the colorants (2) and (3)—this leads to a surprisingly strong bathochromic shift in the absorption and the fluorescence—in addition, the fluorescence is astonishingly intensive; see FIG. 4. As a result, these colorants can be used as NIR colorants: the customary applications as an invisible fluorescent label or as a contrast agent in medicine should be considered here, since the tissue absorbs only a little light in this region.

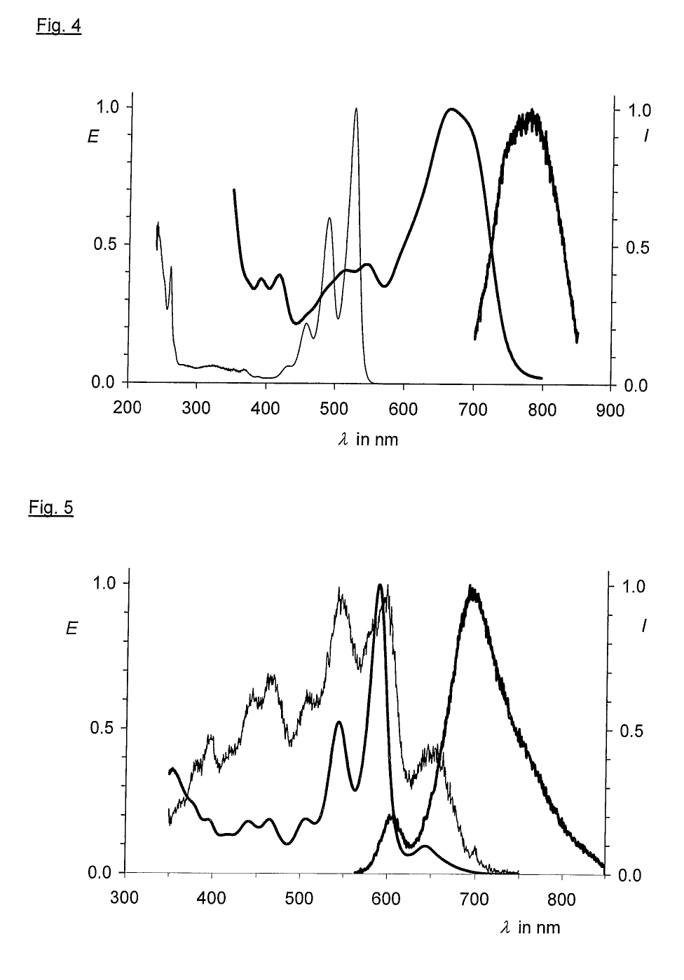

It would be of interest for various practical applications of the colorants only to shift the fluorescence of (2) or (3) to a longer wavelength and to leave the light absorption as in the spectrum. This raises the question of whether this can be brought about by a weak base. We therefore mixed the colorant (2) with the weak base piperidine and achieved an astonishingly large Stokes shift; see FIG. 5. Apparently, the optical excitation of the colorant leads to a sufficiently large rise in the acidity of the N—H group that it can be deprotonated by the weak base, and the astonishingly large Stokes shift thus occurs. The increase in the Stokes shift is accounted for by the ESPT mechanism according to FIG. 5, in which the ground state of the colorant absorbs light and is optically excited as a result. This causes deprotonation of the nitrogen with retention of the optical excitation. The deprotonated species fluoresces with a corresponding long-wave shift, as is the case for the fluorescence of the anion. The ground state attained as a result of the fluorescence is then likewise deprotonated and reverts back to the starting state as a result of protonation; what is astonishing about the cycle process is that it also proceeds so efficiently at the long wavelengths in nonaqueous media, whereas the first ESPT process found by Förster and Weller proceeded in the short-wave spectral region and in an aqueous medium in which the proton transfers are very rapid. The result, a fluorescent colorant with an extremely large Stokes shift in the long-wave spectral region, is extremely interesting for many applications since no fluorescent light is reabsorbed again, as is the case for the conventional fluorescent colorants. This is important especially for applications as laser colorants or as colorants for fluorescent solar collectors.

The situation becomes even more extreme and surprising in the case of colorant (3), since the emission here is shifted far into the NIR region—in this property, the inventive colorants are superior to known materials.

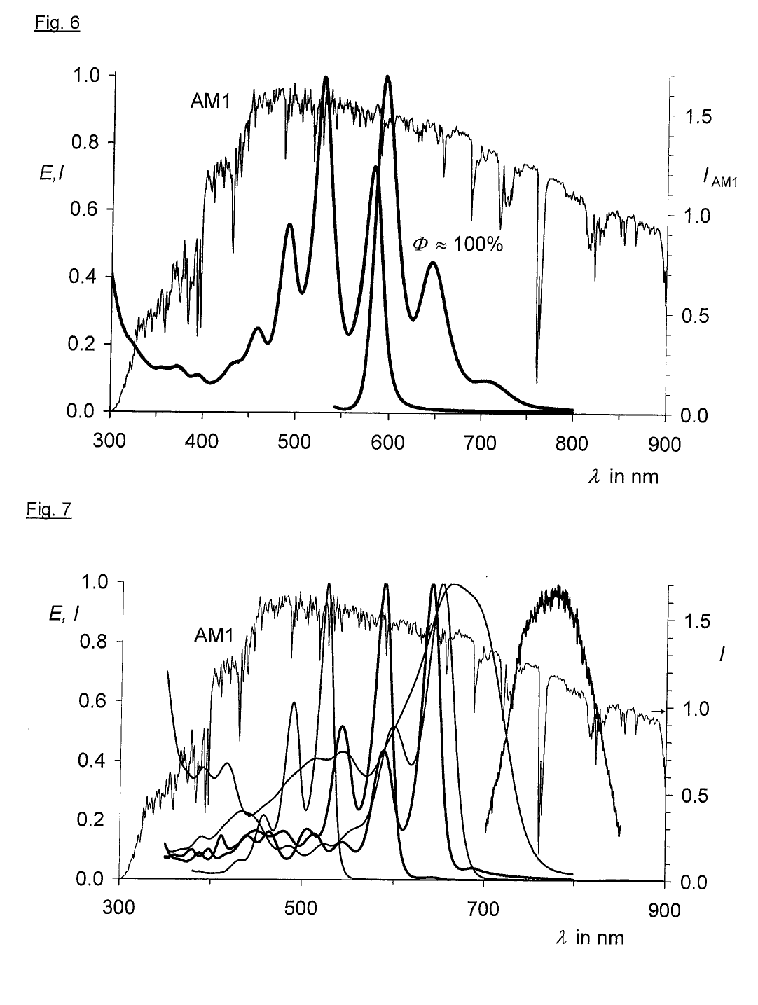

The substances (2) and (3), especially the latter, are of extreme interest for fluorescent solar collectors, since there is a combination of broadband absorption in the visible and strong fluorescence in the NIR region; an impression thereof is given by a comparison with the AM1 solar spectrum according to FIG. 6. When a stack of plates one on top of another is used, the solar spectrum can be separated into individual regions and utilized individually. By means of the ESPT mechanism, it is additionally possible to achieve a large Stokes shift by which absorption and fluorescence spectra are separated spectrally, such that reabsorption of the fluorescent light in the fluorescent solar collector is suppressed. Since the colorants are additionally extremely lightfast, the material is an ideal material for the fluorescent solar collector. When employed as a laser colorant, the broad light absorption is of interest, since optical excitation is possible in this case with broadband-emitting light sources such as flashlamps, since the colorants, by virtue of their broadband absorption, can absorb a large portion of the light and convert it to the spectrally narrower region of the fluorescence.

Colorants such as (2) or (3) can be used in photovoltaics directly in organic solar cells, or else in electrolyte systems, for example the Grätzel cell [Angew. Chem. 2007, 119, 8510-8514]. Finally, colorants of the chromophore (2) are of interest in the cases in which darkening is desired without attenuating the infrared radiation; these colorants are particularly suitable here because they can completely absorb the visible light but not attenuate the infrared light. This is of significance for passive solar heating systems.

The long-wave and broadband absorption of the colorants of the chromophore (3) is of interest when, for example, tinting of glass panes is desired, such as tinting to prevent heating by light radiation. In contrast to tinting with a solely light-absorbing substance, the absorbed light is not simply converted to heat but emitted again as fluorescent light. This heats the absorber to a considerably lesser degree than if a customary nonfluorescent colorant were used.

The novel colorants (2) and (3) can also be used as pigment or textile dyes in conventional technology. In this context, the long-wave light absorption thereof is of interest, through which particular color effects can be achieved.

The invention therefore relates to the following subjects:

1. Diazepinoperylenebisimides of the general formula

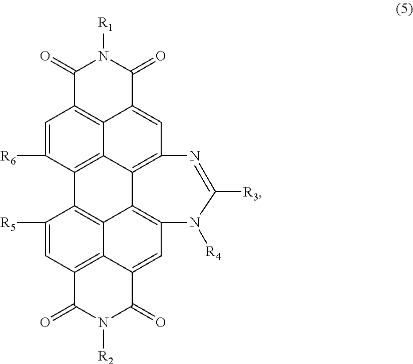

in which the R₁ to R₆ radicals may be the same or different and are each independently hydrogen or linear alkyl radicals having at least one and at most 37 carbon atoms, in which one to 10 CH₂ units may each independently be replaced by carbonyl groups, oxygen atoms, sulfur atoms, selenium atoms, tellurium atoms, cis or trans —CH═CH— groups in which one CH unit may also be replaced by a nitrogen atom, acetylenic C≡C groups, 1,2-, 1,3- or 1,4-substituted phenyl radicals, 2,3-, 2,4-, 2,5-, 2,6-, 3,4- or 3,5-disubstituted pyridine radicals, 2,3-, 2,4-, 2,5- or 3,4-disubstituted thiophene radicals, 1,2-, 1,3-, 1,4-, 1,5-, 1,6-, 1,7-, 1,8-, 2,3-, 2,6- or 2,7-disubstituted naphthalene radicals in which one or two CH groups may be replaced by nitrogen atoms, 1,2-, 1,3-, 1,4-, 1,5-, 1,6-, 1,7-, 1,8-, 1,9-, 1,10-, 2,3-, 2,6-, 2,7-, 2,9-, 2,10- or 9,10-disubstituted anthracene radicals in which one or two CH groups may be replaced by nitrogen atoms. Up to 12 individual hydrogen atoms of the CH₂ groups may each independently also be replaced on the same carbon atoms by the halogens fluorine, chlorine, bromine or iodine, or the cyano group or a linear alkyl chain having up to 18 carbon atoms, in which one to 6 CH₂ units may independently be replaced by carbonyl groups, oxygen atoms, sulfur atoms, selenium atoms, tellurium atoms, cis or trans —CH═CH— groups in which one CH unit may also be replaced by a nitrogen atom, acetylenic C≡C groups, 1,2-, 1,3- or 1,4-substituted phenyl radicals, 2,3-, 2,4-, 2,5-, 2,6-, 3,4- or 3,5-disubstituted pyridine radicals, 2,3-, 2,4-, 2,5- or 3,4-disubstituted thiophene radicals, 1,2-, 1,3-, 1,4-, 1,5-, 1,6-, 1,7-, 1,8-, 2,3-, 2,6- or 2,7-disubstituted naphthalene radicals in which one or two carbon atoms may be replaced by nitrogen atoms, 1,2-, 1,3-, 1,4-, 1,5-, 1,6-, 1,7-, 1,8-, 1,9-, 1,10-, 2,3-, 2,6-, 2,7-, 2,9-, 2,10- or 9,10-disubstituted anthracene radicals in which one or two carbon atoms may be replaced by nitrogen atoms. Up to 12 individual hydrogen atoms of the CH₂ groups of the alkyl radicals may each independently also be replaced on the same carbon atoms by the halogens fluorine, chlorine, bromine or iodine, or cyano groups or linear alkyl chains having up to 18 carbon atoms, in which one to 6 CH₂ units may independently be replaced by carbonyl groups, oxygen atoms, sulfur atoms, selenium atoms, tellurium atoms, cis or trans —CH═CH— groups in which one CH unit may also be replaced by a nitrogen atom, acetylenic C≡C groups, 1,2-, 1,3- or 1,4-substituted phenyl radicals, 2,3-, 2,4-, 2,5-, 2,6-, 3,4- or 3,5-disubstituted pyridine radicals, 2,3-, 2,4-, 2,5- or 3,4-disubstituted thiophene radicals, 1,2-, 1,3-, 1,4-, 1,5-, 1,6-, 1,7-, 1,8-, 2,3-, 2,6- or 2,7-disubstituted naphthalene radicals in which one or two carbon atoms may be replaced by nitrogen atoms, 1,2-, 1,3-, 1,4-, 1,5-, 1,6-, 1,7-, 1,8-, 1,9-, 1,10-, 2,3-, 2,6-, 2,7-, 2,9-, 2,10- or 9,10-disubstituted anthracene radicals in which one or two carbon atoms may be replaced by nitrogen atoms. Instead of bearing substituents, the free valencies of the methine groups or the quaternary carbon atoms may be joined in pairs, so as to form rings, for example cyclohexane rings. The R₁ to R₁₆ radicals may also each independently be the halogen atoms F, Cl, Br or I.

When R₁ to R₆ are hydrocarbon radicals, for example unsubstituted or substituted, unbranched or branched, optionally mono- or polycyclic hydrocarbon radicals, they comprise especially from 1 to 60, preferably from 1 to 37 and more preferably from 1 to 18 carbon atoms. This is especially true of R₁ and R₂. R₃ is preferably phenyl which is unsubstituted or substituted by 1 to 5 identical or different substituents. R₄ is preferably C₁-C₈alkyl or C₃-C₈cycloalkyl each unsubstituted or mono- or polysubstituted by identical or different substituents. R₅ and R₆ are preferably each H.

2. Bisdiazepinoperylenebisimides of the general formula

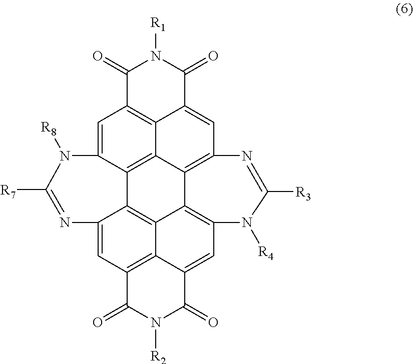

in which all R₁ to R₈ radicals are each as defined in formula (5) for R₁ to R₆.

When R₁ to R₆ are hydrocarbon radicals, for example unsubstituted or substituted, unbranched or branched, optionally mono- or polycyclic hydrocarbon radicals, they comprise especially from 1 to 60, preferably from 1 to 37 and more preferably from 1 to 18 carbon atoms. This is especially true of R₁ and R₂. R₃ and R₇ are preferably each independently phenyl which is unsubstituted or substituted by 1 to 5 identical or different substituents. R₄ and R₈ are preferably each independently C₁-C₈alkyl or C₃-C₈cycloalkyl each unsubstituted or mono- or polysubstituted by identical or different substituents.

3. A process wherein the starting materials used for the preparation of (5) and/or (6) are perylenetetracarboximides, aromatic nitriles and strong bases. Examples of strong bases are sodium amide, potassium hydroxide, potassium hydride and sodium hydride. Examples of aromatic nitriles (aryl nitriles) are benzonitrile, 1- or 2-naphthonitrile, 4-bromobenzonitrile and 4-methoxybenzonitrile.

4. A process wherein the compounds (5) and (6) are synthesized in the presence of oxygen, preferably atmospheric oxygen.

5. A process wherein the perylenetetracarboximide, aryl nitrile and base reactants are effected in substance, preferably at elevated temperature, for example at temperatures between 100 and 200° C., preferably at 160° C.; in this case, the aryl nitrile is used in an equimolecular amount or in excess, preferably in a twofold excess—though it is also possible to use larger excesses. Examples of are benzonitrile, 1- or 2-naphthonitrile, 4-bromobenzonitrile and 4-methoxybenzonitrile.

6. A process wherein the reactants are reacted using solvents. Examples of solvents are ethylene glycol dimethyl ether (glyme) or diethylene glycol dimethyl ether (diglyme).

7. A process wherein the colorants (5) where R₄=H and (6) where R₅ or R₆=H or R₅ and R₆=H are alkylated, preferably methylated, to give the colorants (5) where R₄=alkyl and (6) where R₅ or R₆=alkyl or R₅ and R₆=alkyl radicals. Examples of alkylating agents are alkyl halides with the radicals in question, for example alkyl chlorides, alkyl bromides and alkyl iodides, or mono- and dialkyl sulfates such as dimethyl sulfate, or alkyl/aryl sulfonates such as methyl tosylate. Preferred media for such alkylations are dipolar-aprotic solvents such as DMSO, DMF, N-methylpyrrolidone (NMP), tetramethylurea, DMPU, DMEU or sulfolane.

8. A process wherein the colorants (5) where R₄=H or (6) where R₄ or R₈=H or R₄ and R₈=H are used with addition of weak bases to obtain large Stokes shifts by the ESPT mechanism. Examples of weak bases are amines such as piperidine.

9. The use of the substances (5) or (6) as colorants, preferably as pigments.

10. The use of the substances (5) or (6) as colorants, preferably as pigments for distempers and related colors such as watercolors and inks for inkjet printers, paper inks, printing inks, solventborne and waterborne inks, and other inks for painting and writing purposes, and in paints.

11. The use of the substances (5) or (6) as colorants, preferably as pigments in coating materials. Preferred coating materials are synthetic resin coating materials such as acrylic or vinyl resins, polyester coating materials, novolacs, nitrocellulose coating materials (nitro coating materials), or else natural substances such as zapon lacquer, shellac or qi lacquer (Japan lacquer or China lacquer or East Asian lacquer).

12. The use of the colorants (5) or (6) in data stores, preferably in optical stores. Examples are systems such as the CD or DVD.

13. The use of the substances (5) or (6) as fluorescent colorants.

14. The use of the substances (5) or (6) in OLEDs (organic light-emitting diodes).

15. The use of the substances (5) or (6) in photovoltaic systems.

16. The employment of the colorants (5) or (6) for bulk coloring of polymers. Examples are materials composed of polyvinyl chloride, polyvinylidene chloride, polyacrylic acid, polyacrylamide, polyvinyl butyral, polyvinylpyridine, cellulose acetate, nitrocellulose, polycarbonates, polyamides, polyurethanes, polyimides, polybenzimidazoles, melamine resins, silicones such as polydimethylsiloxane, polyesters, polyethers, polystyrene, polydivinylbenzene, polyvinyltoluene, polyvinylbenzyl chloride, polymethyl methacrylate, polyethylene, polypropylene, polyvinyl acetate, polyacrylonitrile, polyacrolein, polybutadiene, polychlorobutadiene or polyisoprene, or the copolymers of the monomers mentioned.

17. The employment of the colorants (5) or (6) for coloring of natural substances. Examples are paper, wood, straw, or natural fiber materials such as cotton, jute, sisal, hemp, flax or/and the conversion products thereof, for example viscose fibers, nitrate silk, or cuprammonium rayon.

18. The employment of the colorants (5) or (6) as mordant dyes, for example for coloring of natural substances. Examples are paper, wood, straw, or natural fiber materials such as cotton, jute, sisal, hemp, flax or the conversion products thereof, for example viscose fibers, nitrate silk or cuprammonium rayon. Preferred salts for dipping are aluminum, chromium and iron salts.

19. The employment of the colorants (5) or (6) as colorants, for example for coloring of inks, coating materials and other paints, paper inks, printing inks, solventborne inks and other inks for writing and painting purposes.

20. The employment of the colorants (5) or (6) as pigments in electrophotography: for example for dry copier systems (Xerox process) and laser printers (“non-impact printing”).

21. The employment of the colorants (5) or (6) for security marking purposes, in which case the great chemical and photochemical stability and in some cases also the fluorescence of the substances is of significance. This is preferably for checks, check cards, banknotes, coupons, documents, identification papers and the like, in which a particular, unmistakable color impression is to be achieved.

22. The employment of the colorants (5) or (6) as an addition to other colors, in which a particular color shade is to be achieved, preference being given to particularly luminous hues.

23. The employment of the colorants (5) or (6) for marking articles for machine recognition of these articles via the fluorescence, preference being given to the machine recognition of articles for sorting, for example including for the recycling of plastics.

24. The employment of the colorants (5) or (6) as a fluorescent colorant for machine-readable markings, preference being given to alphanumeric impressions or barcodes.

25. The employment of the colorants (5) or (6) for frequency conversion of light, for example in order to convert short-wave light to longer-wave visible light.

26. The employment of the colorants (5) or (6) in display elements for all kinds of display, information and marking purposes, for example passive display elements, and information and traffic signs, such as traffic lights.

27. The employment of the colorants (5) or (6) in inkjet printers in homogeneous solution as a fluorescent ink.

28. The employment of the colorants (5) or (6) as a starting material for superconductive organic materials.

29. The employment of the colorants (5) or (6) for solid fluorescent markings.

30. The employment of the colorants (5) or (6) for decorative purposes.

31. The employment of the colorants (5) or (6) for artistic purposes.

32. The employment of the colorants (5) or (6) for tracer purposes, for example in biochemistry, medicine, technology and natural science. It is possible here for the colorants to be covalently bonded to substrates or via secondary valencies such as hydrogen bonds or hydrophobic interactions (adsorption).

33. The employment of the colorants (5) or (6) as fluorescent colorants in high-sensitivity detection processes.

34. The employment of the colorants (5) or (6) as fluorescent colorants in scintillators.

35. The employment of the colorants (5) or (6) as colorants or fluorescent colorants in optical light-collecting systems.

36. The employment of the colorants (5) or (6) as colorants or fluorescent colorants in fluorescent solar collectors.

37. The employment of the colorants (5) or (6) as colorants or fluorescent colorants in fluorescence-activated displays.

38. The employment of the colorants (5) or (6) as colorants or fluorescent colorants in cold light sources for light-induced polymerization to prepare polymers.

39. The employment of the colorants (5) or (6) as colorants or fluorescent colorants for material testing, for example in the production of semiconductor circuits.

40. The employment of the colorants (5) or (6) as colorants or fluorescent colorants for the examination of microstructures of integrated semiconductor components.

41. The employment of the colorants (5) or (6) as colorants or fluorescent colorants in photoconductors.

42. The employment of the colorants (5) or (6) as colorants or fluorescent colorants in photographic processes.

43. The employment of the colorants (5) or (6) as colorants or fluorescent colorants in display, illumination or image converter systems in which the excitation is effected by electrons, ions or UV radiation, for example in fluorescent displays, Braun tubes or in luminescent tubes.

44. The employment of the colorants (5) or (6) as colorants or fluorescent colorants as part of an integrated semiconductor circuit, the colorants as such or in conjunction with other semiconductors, for example in the form of an epitaxy.

45. The employment of the colorants (5) or (6) as colorants or fluorescent colorants in chemiluminescent systems, for example in chemiluminescent glow sticks, in luminescence immunoassays or other luminescence detection processes.

46. The employment of the colorants (5) or (6) as colorants or fluorescent colorants as signal colors, preferably for visual emphasis of inscriptions and drawings or other graphic products, for identifying signs and other objects in which a particular visual color impression is to be achieved.

47. The employment of the colorants (5) or (6) as colorants or fluorescent colorants in dye lasers, preferably as fluorescent colorants for generating laser beams.

48. The employment of the colorants (5) or (6) as colorants in dye lasers as Q-switches.

49. The employment of the colorants (5) or (6) as active substances for nonlinear optics, for example for the frequency doubling and the frequency tripling of laser light.

50. The employment of the colorants (5) or (6) as rheology improvers.

51. The employment of the colorants (5) or (6) for leak testing of closed systems.

52. A process for inducing fluorescence in the range from 500 to 1000 nm, which comprises irradiating a colorant (5) or (6) with electromagnetic radiation of wavelength from 250 to 600 nm, preferably with visible light of wavelength from 400 to 600 nm. The fluorescence generated as a result can be used, for example, to generate power or heat, or to conduct a chemical reaction.

53. A process for detecting fluorescence in the range from 500 to 1000 nm, which comprises inducing the fluorescence by irradiating a colorant (5) or (6) with electromagnetic radiation of wavelength from 250 to 600 nm, preferably visible light of wavelength from 400 to 600 nm. The detected fluorescence can be partly or completely collected and converted to analog or digital signals or to energy.

The processes and uses disclosed above under points 3 to 53 also apply to the further isomeric structures disclosed hereafter in points 54 to 60, which are obtainable in the same way.

54. Imidazoloperylenebisimides of the general formula

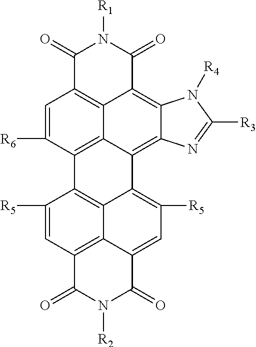

in which the R₁ to R₆ radicals are each as defined for formula (5) or (6).

55. Imidazoloperylenebisimides of the general formula

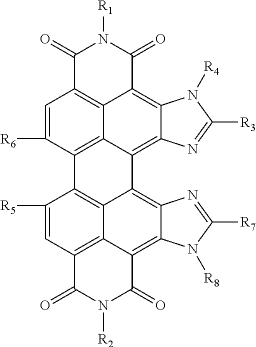

in which the R₁ to R₈ radicals are each as defined for formula (5) or (6).

56. Imidazoloperylenebisimides of the general formula

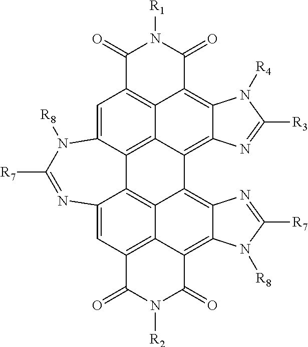

in which the R₁ to R₅ radicals are each as defined for formula (5) or (6).

57. Imidazoloperylenebisimides of the general formula

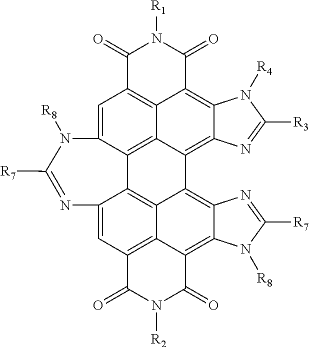

in which the R₁ to R₈ radicals are each as defined for formula (5) or (6).

58. Imidazoloperylenebisimides of the general formula

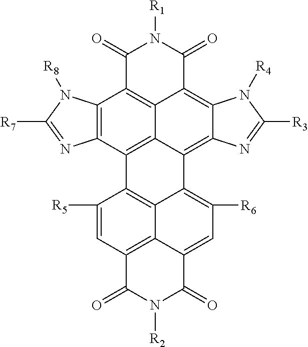

in which the R₁ to R₈ radicals are each as defined for formula (5) or (6).

59. Imidazoloperylenebisimides of the general formula

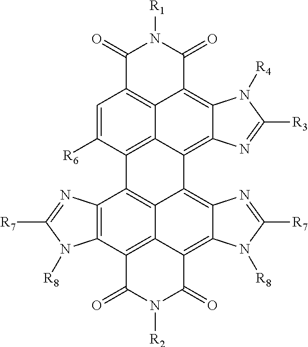

in which the R₁ to R₈ radicals are each as defined for formula (5) or (6).

60. Imidazoloperylenebisimides of the general formula

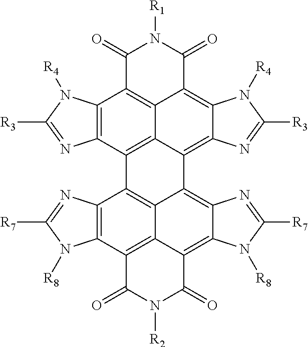

in which the R₁ to R₈ radicals are each as defined for formula (5) or (6).

FIG. 1 shows the synthesis scheme of the diazepinoperylenetetracarboximides.

FIG. 2 shows the UV/Vis absorption (thick line to the left) and fluorescence spectra (thick line to the right) of (2a) in chloroform compared to the absorption spectrum of (1a) (thin line).

FIG. 3 shows the UV/Vis absorption (thick line, to the left) and fluorescence spectra (thick line to the right) of (3a) in chloroform compared to the absorption spectrum of (1a) (thin line).

FIG. 4 shows the UV/Vis absorption (thick line, to the left) and fluorescence spectra (thick line to the right) of (2a) in chloroform with addition of DBU compared to the absorption spectrum of (1a) (thin line).

FIG. 5 shows the ESPT mechanism of 2a with the absorption spectrum (thick line to the left), the fluorescence spectrum (thick line to the right) and the fluorescence excitation spectrum of (2a) at 694 nm in 3.1:1 chloroform/piperidine.

FIG. 6 shows the broadband absorption and fluorescence spectrum of the colorant according to Example 25 in chloroform.

FIG. 7 shows an overview of the UV/Vis absorption spectra in chloroform. The maxima correspond, from left to right, to the compound (1a), to the compound according to Example 18, to the compound according to Example 22, to the compound according to Example 25, to the deprotonated form of the compound according to Example 18 and to the fluorescence spectrum of the deprotonated form of the compound according to Example 18 compared to the AM1 solar spectrum (noisy upper line).

The preceding text has discussed principally only the structures (1) to (6). However, it should be mentioned that it was not possible to elucidate the structures of the inventive colorants with absolute certainty. Structures (2) to (6) therefore reflect only one of several possible interpretations of the experimental analytical data. The methods nowadays available do not allow entirely satisfactory assignment of the structure. However, the substructures thereof are assured, which can be represented as follows:

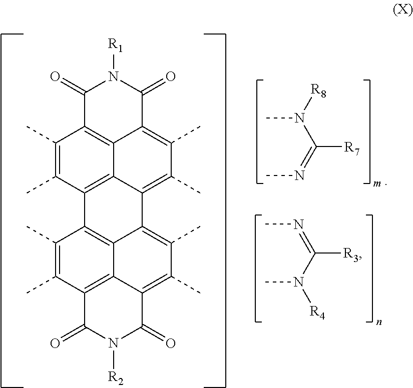

In formula (X), m is 0, 1 or 2 and n is 1 or 2. The substituents are of course the same as disclosed above. The exact definition thereof is disclosed once again in claim 1.

The examples which follow, however, also give clear indications to the following additional structures, if appropriate including the isomers thereof with regard to the exact position of R₄ and R₈ on the two nitrogen atoms of the imidazole rings (or tautomers when R₄ and/or R₈ are each H):

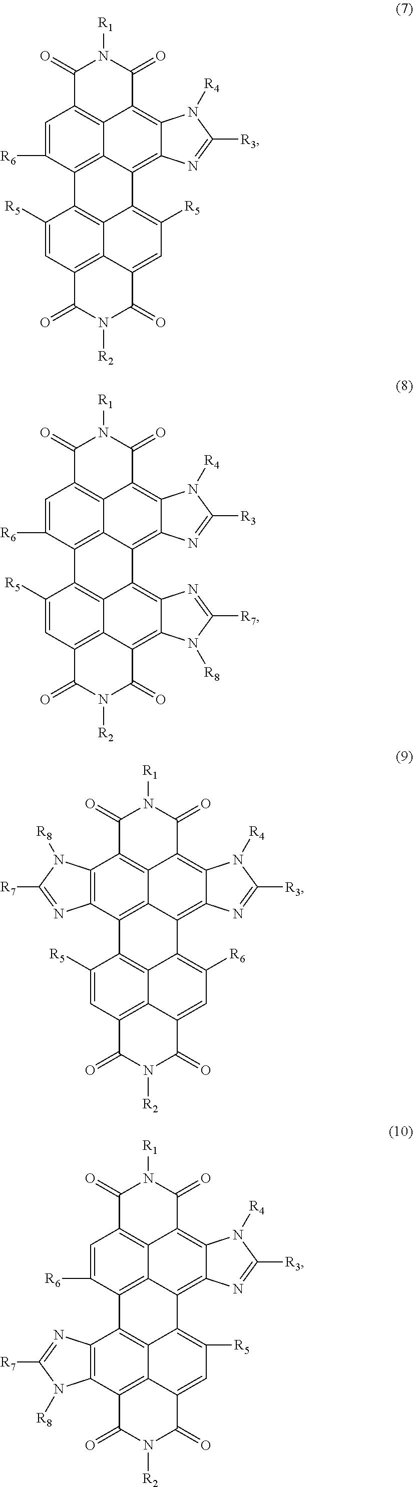

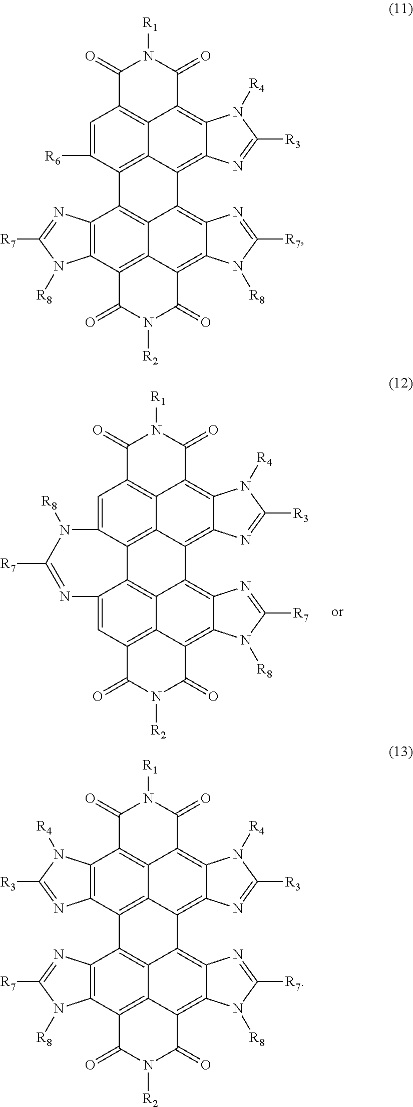

The inventive compounds can alternatively also be obtained by the condensation of an amide-substituted perylenediimide in the presence of a strong base (as before, for example, sodium amide), optionally in the presence of a solvent.

The examples which follow illustrate the invention without restricting the scope thereof (where not stated otherwise, “%” is always % by weight):

### General:

IR spectra: Perkin Elmer 1420 Ratio Recording Infrared Spektrometer, FT 1000; UV/Vis spectra: Varian Cary 5000 and Bruins Omega 20; fluorescence spectra: Perkin Elmer FS 3000 (totally corrected); NMR spectroscopy: Varian Vnmrs 600 (600 MHz); mass spectrometry: Finnigan MAT 95.

### EXAMPLE 1

2,10-Bis(1-hexylheptyl)-6-phenyl[1,3]diazepino[4′,5′,6′:4,5]phenanthro-[2,1,10-def::7,8,9-d′e′f′]diisoquinoline-1,3,9,11(2H,5H,9H,11H)-tetraone (2a) and 2,9-bis(1-hexylheptyl)bis[1,3]diazepino[4′,5′,6′:1,12;4″,5″,6″:6,7]perylo[3,4-cd:9,10-c′d′]dipyridine-1,3,9,11(2H,5H,10H,13H)-tetraone (3a). N,N′-Bis(1-hexylheptyl)-perylene-3,4:9,10-tetracarboximide ((1a), 1.00 g, 1.32 mmol) and NaNH₂ (1.00 g, 25.6 mmol) are suspended in benzonitrile (250 mL), heated to 165° C. for 3 h (blue color), allowed to cool and extracted by shaking with a 1:1 mixture of 2N HCl and CHCl₃ (300 mL). The organic phase was dried (MgSO₄), concentrated by evaporation to dryness under reduced pressure (removal of excess benzonitrile), taken up in CHCl₃, filtered and purified by column chromatography (silica gel, CHCl₃). A single adduct and a double adduct of benzonitrile are obtained. 1^{st} fraction: 2,9-Bis(1-hexylheptyl)bis[1,3]diazepino[4′,5′,6′:1,12;4″,5″,6″:6,7]perylo[3,4-cd:9,10-c′d′]dipyridine-1,3,9,11(2H,5H,10H,13H)-tetraone (3a). Yield: 40 mg (4%) of black colorant; m.p. >300° C.; R_{f} (silica gel, chloroform)=0.88; IR (ATR): \tilde{v}=3316.7 m, 2950.3 m, 2920.1 s, 2852.0 m, 1672.9 s, 1616.9 s, 1586.9 s, 1532.3 w, 1487.0 w, 1453.1 w, 1436.5 w, 1401.4 w, 1339.6 m, 1332.0 s, 1299.4 w, 1267.0 w, 1232.4 w, 1184.3 w, 1109.1 w, 1058.6 m, 1000.6 m, 955.5 w, 895.7 w, 867.9 w, 815.0 w, 779.6 w, 758.2 m, 731.6 w, 687.6 w cm⁻¹; ¹H NMR (600 MHz, CDCl₃, 25° C.), δ=0.82-0.89 (m, 12H, CH₃), 1.26-1.47 (m, 32H, CH₂), 1.91-2.06 (dm, 4H, β-CH₂), 2.28-2.43 (m, 4H, β-CH₂), 5.22-5.36 (m, 2H, CH—N), 7.65-7.67 (m, 3H, CH_{aryl}), 8.33-8.38 (m, 2H, CH_{aryl}), 8.72-8.85 (m, 6H, H_{pery}), 10.95 (d, 1H, ³J=8.0 Hz), 11.55 ppm (s, 1H, N—H); ¹³C NMR (151 MHz, CDCl₃, 25° C.): δ=14.1, 22.6, 27.0, 29.3, 31.8, 32.4, 54.6, 104.8, 121.2, 122.7, 123.5, 126.5, 126.6, 127.7, 128.1, 129.1, 129.4, 130.4, 130.6, 131.1, 132.2, 134.7, 134.9, 135.2, 135.2, 138.7, 39.0, 139.2, 143.9, 157.3, 163.9, 164.9, 165.7 ppm; UV/Vis (CHCl₃): λₘₐₓ (Eᵣₑₗ)=388.6 (0.07), 413.0 (0.12), 452.6 (0.14), 481.0 (0.15), 514.4 (0.14), 547.2 (0.11), 589.6 (0.42), 640.6 (1.00); fluorescence (CHCl₃): λₘₐₓ (Iᵣₑₗ)=650.8 (1.00), 712.3 nm, (0.32), fluorescence quantum yield (CHCl₃, λ_{exc}=472 nm, E_{472 nm}=0.149 cm⁻¹, reference: S13 at 1.00): 1.00; MS (DEI⁺/70 eV): m/z (%)=990 (25) [M⁺+4H], 989 (63) [M⁺+3H], 988 (91) [M⁺+4H], 806 (19), 805 (20), 624 (21), 623 (66), 622 (100), 55 (11); HMRS(C₆₄H₇₁N₆O₄): calc. m/z: 987.554; found m/z: 987.552, Δ=−2 mmu.

2^{nd} Fraction: Yield 409 mg (36%) of 2,10-bis(1-hexylheptyl)-6-phenyl[1,3]diazepino-[4′,5′,6′:4,5]phenanthro[2,1,10-def::7,8,9-d′e′f′]diisoquinoline-1,3,9,11(2H,5H,9H,-11H)-tetraone (2a) as a metallically shiny, violet colorant, m.p. >300° C., R_{f} (silica gel, chloroform)=0.85, IR (ATR): \tilde{v}=3411.7 m, 2954.8 m, 2923.5 s, 2855.2 m, 1689.1 s, 1656.0 s, 1640.3 s, 1623.0 s, 1591.7 s, 1534.8 w, 1486.6 w, 1455.4 w, 1432.6 w, 1396.1 w, 1375.6 w, 1342.4 s, 1332.0 s, 1305.6 m, 1257.4 m, 1219.9 w, 1179.1 w, 1090.9 m, 1054.9 m, 1016.0 m, 871.0 w, 807.6 m, 796.5 m, 748.1 m, 727.0 w, 684.0 w cm⁻¹; ¹H NMR (600 MHz, CDCl₃, 25° C.): δ=0.81-0.85 (m, 12H, CH₃), 1.23-1.40 (m, 32H, CH₂), 1.86-1.97 (m, 4H, β-CH₂), 2.24-2.37 (m, 4H, β-CH₂), 5.18-5.30 (m, 2H, CH—N), 7.67-7.69 (m, 3H, CH_{aryl}), 8.33-8.35 (m, 2H, CH_{aryl}), 8.56-8.77 (m, 6H, H_{pery}), 10.74 (d, 1H, ³J=8.2 Hz), 11.52 ppm (s, 1H, N—H), ¹³C NMR (151 MHz, CDCl₃, 25° C.): δ=14.1, 22.6, 27.0, 29.3, 31.8, 32.4, 54.6, 104.8, 121.2, 122.7, 123.5, 126.5, 126.6, 127.7, 128.1, 129.1, 129.4, 130.4, 130.6, 131.1, 132.2, 134.7, 134.9, 135.2, 135.2, 138.7, 39.0, 139.2, 143.9, 157.3, 163.9, 164.9, 165.7 ppm; UV/Vis (CHCl₃): λₘₐₓ (Eᵣₑₗ)=378.6 (9020), 396.9 (9210), 439.4 (13400), 463.6 (14700), 504.6 (15900), 541.9 (48100), 586.6 nm (92000); fluorescence (CHCl₃): λₘₐₓ (Iᵣₑₗ)=599.3 (1.00), 651.5 (0.43); fluorescence quantum yield (CHCl₃, λ_{exc}=541 nm, E_{541 nm}=0.322 cm⁻¹, reference: (1a) at Φ=1.00): 1.00; HMRS(C₅₇H₆₇N₄O₄): calc. m/z: 871.516; found m/z: 871.517; Δ=1 mmu; C₅₇H₆₇N₄O₄ (871.2): calc. C, 78.59%; H, 7.64; N, 6.43%. found C, 78.49; H, 7.78; N, 6.55%.

### EXAMPLE 2

2,10-Bis(1-hexylheptyl)-6-(2-naphthyl)[1,3]diazepino[4′,5′,6′:4,5]-phenanthro[2,1,10-def::7,8,9-d′e′f′]diisoquinoline-1,3,9,11(2H,5H,9H,11H)-tetraone (2e). N,N′-Bis(1-hexylheptyl)perylene-3,4:9,10-tetracarboximide ((1a), 200 mg, 0.264 mmol), NaNH₂ (1.00 g, 5.12 mmol) and 2-naphthonitrile (8 g) were converted and worked up as in Example 1. Yield: 54 mg (22%) of a violet colorant, m.p. >300° C.; R_{f} (silica gel, chloroform)=0.90; IR (ATR): \tilde{v}=3409.1 m, 2953.2 m, 2922.7 s, 2853.1 m, 1690.6 s, 1655.7 s, 1640.2 s, 1624.2 s, 1607.6 w, 1591.4 s, 1527.4 w, 1507.9 w, 1458.7 w, 1432.3 w, 1411.5 w, 1379.2 w, 1343.4 s, 1343.4 m, 1248.2 m, 1220.4 w, 1179.01 10 w, 1120.0 wm, 974.1 m, 872.0 w, 852.3 m, 810.3 m, 749.3 m, 727.9 w, 632.3 w, 581.2 w cm⁻¹; ¹H NMR (300 MHz, CDCl₃, 25° C.): δ=0.81-0.87 (m, 12H, CH₃), 1.27-1.50 (m, 32H, CH₂), 1.89-2.04 (m, 4H, β-CH₂), 2.26-2.44 (m, 4H, β-CH₂), 5.20-5.34 (m, 2H, CH—N), 7.52-7.64 (m, 2H, CH_{aryl}), 7.81-8.00 (m, 3H, CH_{aryl}), 8.32-8.37 (m, 2H, CH_{aryl}), 8.46-8.72 (m, 6H, H_{pery}), 10.61 (d, 1H, ³J=8.2 Hz), 11.54 ppm (s, 1H, N—H); ¹³C NMR (151 MHz, CDCl₃, 25° C.): δ=14.1, 22.6, 27.0, 29.3, 31.8, 32.5, 54.6, 120.9, 122.2, 122.4, 123.0, 123.3, 124.0, 124.8, 125.2 126.4, 126.4, 127.2, 127.6, 128.0 128.3, 128.9, 129.0, 129.2, 130.1, 130.4, 130.8, 139.9, 132.9, 134.5, 134.6, 134.62, 139.1, 143.8, 157.1, 163.7, 164.7, 164.8 ppm; UV/Vis (CHCl₃): λₘₐₓ (Eᵣₑₗ)=384.4 (0.14), 451.8 (0.20), 476.6 (0.23), 507.5 (0.19), 545.6 (0.54), 591.2 (1.00); fluorescence (CHCl₃) λmax (Iᵣₑₗ)=603.0 (1.00), 655.5 (0.46); fluorescence quantum yield (CHCl₃, λ_{exc}=472 nm, E_{472 nm}=0.159 cm⁻¹, reference: (1a) at Φ=1.00): 1.00; HMRS(C₆₁H₆₈N₄O₄): calc. m/z: 920.524, found m/z 920.522; Δ=−0.002.

### EXAMPLE 3

2,10-Bis(1-hexylheptyl)-6-(4-bromophenyl)[1,3]diazepino[4′, 5′,6′:4,5]-phenanthro[2,1,10-def::7,8,9-d′e′f′]diisoquinoline-1,3,9,11(2H,5H,9H,11H)-tetraone (2c). N,N′-Bis(1-hexylheptyl)perylene-3,4:9,10-tetracarboximide ((1a), 100 mg, 0.264 mmol), NaNH₂ (1.00 g, 2.56 mmol) and 4-bromobenzonitrile (10 g) were converted and worked up as in Example 1. Yield 97 mg (77%) of violet colorant; m.p. >300° C.; R_{f} (silica gel, chloroform)=0.80, IR (ATR): \tilde{v}=3407.7 m, 2952.5 m, 2923.3 s, 2854.8 m, 1690.2 s, 1641.2 s, 1623.1 s, 1591.6 s, 1531.7 w, 1481.0 w, 1467.0 w, 1434.22.6 w, 1411.3 w, 1387.0 w, 1343.7 s, 1331.0 s, 1255.4 m, 1219.6 w, 1179.8 w, 1119.8 w, 1071.9 w, 1008.7 m, 871.3 w, 809.0 m, 772.0 w, 748.5 m, 724.1 w, 596.2 w cm⁻¹; ¹H NMR (600 MHz, CDCl₃, 25° C.): δ=0.81-0.89 (m, 12H, CH₃), 1.22-1.39 (m, 32H, CH₂), 1.87-1.97 (m, 4H, β-CH₂), 2.25-2.36 (m, 4H, β-CH₂), 5.18-5.31 (m, 2H, CH—N), 7.81-7.83 (m, 2H, CH_{aryl}), 8.23-8.25 (m, 2H, CH_{aryl}), 8.65-8.84 (m, 6H, H_{pery}), 10.78 (d, 1H, ³J=8.2 Hz), 11.52 ppm (s, 1H, N—H); ¹³C NMR (151 MHz, CDCl₃, 25° C.): δ=14.1, 22.6, 27.0, 29.3, 31.8, 32.4, 54.6, 121.4, 122.8, 123.7, 126.7, 126.7, 127.0, 127.2, 129.1, 129.2, 132.8, 143.9 ppm; UV/Vis (CHCl₃): λₘₐₓ (ε)=378.9 (9540), 399.0 (8870), 441.3 (14100), 465.0 (15400), 505.8 (15000), 542.9 (46600), 587.5 (89400); fluorescence (CHCl₃): λₘₐₓ (Iᵣₑₗ)=599.8 (1.00), 653.5 (0.42); fluorescence quantum yield (CHCl₃, λ_{exc}=495 nm, E_{495 nm}=0.053 cm⁻¹, reference: (1a) at Φ=1.00): 1.00, HMRS(C₅₇H₆₅BrN₄O₄): calc. m/z 950.431, found m/z: 950.429, Δ=−2 mmu; C₅₇H₆₅BrN₄O₄ (950.1): calc. C, 72.06; H, 6.90; N, 5.90. found C, 71.79; H, 6.73; N, 5.88.

### EXAMPLE 4

10-Bis[1-(1-methylethyl)-2-methylpropyl]-6-phenyl[1,3]diazepino-[4′,5′,6′:4,5]phenanthro[2,1,10-def::7,8,9-d′e′f′]diisoquinoline-1,3,9,11(2H,5H,9H,11H)-tetraone (2b). N,N′-Bis[1-(1-methylethyl)-2-methylpropyl]perylene-3,4:9,10-tetracarboximide ((1b), 1.30 g, 1.85 mmol), NaNH₂ (1.30 g, 33.3 mmol) and benzonitrile (250 g) were converted and worked up as in Example 1. Yield 670 mg (43%) of metallically shiny, violet colorant; m.p. >300° C.; R_{f} (silica gel, chloroform)=0.25; IR (ATR): \tilde{v}=3407.6 m, 2962.1 m, 2923.5 m, 2872.3 m, 1698.7 m, 1683.5 m, 1659.4 m, 1639.5 s, 1628.5 s, 1608.2 m, 1591.9 s, 1566.2 w, 1532.8 w, 1487.6 w, 1457.0 w, 1433.1 w, 1411.6 w, 1382.6 w, 1332.0 s, 1306.2 m, 1252.1 m, 1212.4 w, 1167.3 w, 1123.5 w, 1100.3 w, 1052.2 w, 924.4 w, 904.0 w, 872.0 w, 844.1 w, 811.9 m, 775.6 w, 751.3 m, 684.0 w, 585.4 w cm⁻¹; ¹H NMR (600 MHz, CDCl₃, 25° C.): δ=0.97-1.01 (m, 12H, CH₃), 1.14-1.18 (m, 12H, CH₃), 2.72-2.83 (m, 4H, β-CH), 4.77-4.88 (m, 2H, CH—N), 7.67-7.69 (m, 3H, CH_{aryl}), 8.36-8.38 (m, 2H, CH_{aryl}), 8.62-8.84 (m, 6H, H_{pery}), 10.79-10.83 (m, 1H, CH_{pery}), 11.57-11.59 ppm (m, 1H, N—H); ¹³C NMR (151 MHz, CDCl₃, 25° C.): δ=20.7, 21.9, 22.0, 29.2, 29.4, 65.0, 65.1, 121.2, 121.3, 122.3, 122.4, 122.7, 122.8, 123.0, 123.1, 123.6, 125.1, 125.2, 125.3, 125.4, 126.6, 126.7, 127.7, 127.8, 128.2, 129.2, 129.4, 129.5, 130.6, 130.7, 131.3, 131.9, 132.2, 132.6, 134.8, 134.9, 135.0, 135.3, 139.1, 139.4, 144.0, 157.4, 164.2, 164.4, 164.5, 165.4, 165.5, 166.3 ppm; UV/Vis (CHCl₃): λₘₐₓ (Eᵣₑₗ)=379.7 (0.10), 397.5 (0.10), 439.1 (0.15), 463.5 (0.17), 504.4 (0.19), 542.2 (0.52), 586.7 (1.00); fluorescence (CHCl₃): λₘₐₓ (Iᵣₑₗ)=599.0 (1.00), 650.3 (0.43); fluorescence quantum yield (CHCl₃, λ_{exc}=495 nm, E_{495 nm}=0.053 cm⁻¹, reference: (1a) at Φ=1.00): 1.00; HMRS (C₄₅H₄₂N₄O₄): calc. m/z: 702.320, found m/z: 702.319, Δ=−0.001; C₄₅H₄₂N₄O₄ (702.8): calc. C, 76.90; H, 6.02; N, 7.97. found C, 76.55; H, 5.91; N, 7.94.

### EXAMPLE 5

2,10-Bis(1-hexylheptyl)-6-(4-methoxyphenyl)[1,3]diazepino[4′,5′,6′:4,5]-phenanthro[2,1,10-def::7,8,9-d′e′f′]diisoquinoline-1,3,9,11(2H,5H,9H,11H)-tetraone (2d). N,N′-Bis(1-hexylheptyl)perylene-3,4:9,10-tetracarboximide ((1a), 500 mg, 0.660 mmol), NaNH₂ (500 mg, 12.8 mmol) and 4-methoxybenzonitrile (20 g) were converted and worked up as in Example 1. Yield 137 mg (23%) of metallically shiny, violet colorant; m.p. >300° C.; R_{f} (silica gel, chloroform)=0.80; IR (ATR): \tilde{v}=3411.8 m, 2951.8 m, 2921.6 s, 2853.3 m, 1687.4 s, 1654.2 s, 1638.8 s, 1622.0 s, 1608.2 s, 1591.2 s, 1489.7 w, 1466.1 w, 1433.9 w, 1411.8 w, 1393.2 w, 1373.5 w, 1342.3 s, 1329.9 s, 1301.5 m, 1250.6 s, 1219.4 w, 1172.3 w, 1120.0 w, 1058.3 w, 1029.0 w, 840.2 w, 832.2 m, 809.0 m, 749.2 m, 726.8 w cm⁻¹; ¹H NMR (300 MHz, CDCl₃, 25° C.): δ=0.81-0.85 (m, 12H, CH₃), 1.23-1.40 (m, 32H, CH₂), 1.86-1.97 (m, 4H, β-CH₂), 2.24-2.37 (m, 4H, β-CH₂), 3.99 (s, 3H, CH₃), 5.18-5.30 (m, 2H, CH—N), 7.14 (d, 2H, ³J=8.8 Hz, CH_{aryl}), 8.22 (d, 2H, ³J=8.7 Hz, CH_{aryl}), 8.38-8.68 (m, 6H, H_{pery}), 10.57 (d, 1H, ³J=8.1 Hz), 11.30 ppm (s, 1H, N—H); UV/Vis (CHCl₃): λₘₐₓ (ε)=380.0 (7610), 399.0 (7490), 465.1 (15700), 482.1 (19800), 511.8 (16300), 548.1 (41100), 592.7 (73500); fluorescence quantum yield (CHCl₃, λ_{exc}=465 nm, E_{465 nm}=0.00434 cm⁻¹, reference: (1a) at Φ=1.00): 1.00; HMRS(C₅₈H₆₉N₄O₅): calc. m/z: 901.528; found m/z: 901.530, Δ=2 mmu; C₅₈H₆₉N₄O₅ (901.2): calc. C, 77.30; H, 7.61; N, 6.22. found C, 77.34; H, 7.78; N, 6.09%.

### EXAMPLE 6

2,10-Bis(1-hexylheptyl)-5-methyl-6-phenyl[1,3]diazepino[4′,5′,6′:4,5]-phenanthro[2,1,10-def::7,8,9-d′e′f′]diisoquinoline-1,3,9,11(2H,5H,9H,11H)-tetraone (4a). 2,10-Bis(1-hexylheptyl)-6-phenyl[1, 3]diazepino[4′,5′,6′:4,5]phenanthro[2,1,10-def::7,8,9-d′e′f′]diisoquinoline-1,3,9,11(2H,5H,9H,11H)-tetraone (2a) according to Example 1 (100 mg, 0.115 mmol) was dissolved in THF (30 mL) and admixed with aqueous KOH (10%, 2.5 mL), stirred until the color turned blue, admixed dropwise with dimethyl sulfate (0.3 ml, caution: toxic), stirred at room temperature for 3 h, admixed with water (100 mL), filtered off with suction, washed with methanol and purified by column chromatography. Yield 90 mg (88%) of violet colorant, m.p. >300° C., R_{f} (silica gel, chloroform)=0.80; IR (ATR): \tilde{v}=2952.8 m, 2922.3 s, 2854.5 m, 1688.8 s, 1645.9 s, 1588.8 s, 1528.5 w, 1486.3 w, 1462.8 w, 1424.1 w, 1404.1 w, 1332.2 s, 1253.5 m, 1219.9 w, 1179.2 w, 1099.5 w, 1023.2 m, 1016.0 w, 871.3 w, 808.0 m, 772.5 w, 746.0 m, 700.3 w, 600.0 w cm⁻¹; ¹H NMR (300 MHz, CDCl₃, 25° C.): δ=0.81-0.84 (m, 12H, CH₃), 1.23-1.39 (m, 32H, CH₂), 1.87-1.93 (m, 4H, β-CH₂), 2.26-2.33 (m, 4H, β-CH₂), 4.01 (s, 3H, CH₃), 5.19-5.24 (m, 2H, CH—N), 7.67-7.72 (m, 3H, CH_{aryl}), 8.09-8.10 (m, 2H, CH_{aryl}), 8.56-8.72 (m, 6H, H_{pery}), 10.74 ppm (d, 1H, ³J=8.2 Hz); ¹³C NMR (151 MHz, CDCl₃, 25° C.): δ=14.0, 22.6, 27.0, 29.3, 31.8, 32.4, 39.0, 54.6, 121.1, 121.1, 122.4, 122.4, 123.4, 126.7, 127.7, 129.0, 129.0, 129.1, 130.4, 131.0, 131.4, 134.6, 144.2, 163.0, 163.9, 165.0 ppm; UV/Vis (CHCl₃): λₘₐₓ (Eᵣₑₗ)=377.5 (0.11), 394.5 (0.11), 437.6 (0.13), 502.9 (0.18), 540.2 (0.53), 584.8 (1.00); fluorescence (CHCl₃): λₘₐₓ (Iᵣₑₗ)=599.0 (1.00), 650.0 (0.44); fluorescence quantum yield (CHCl₃, λ_{exc}=490 nm, E_{490 nm}=0.0094 cm⁻¹, reference: (1a) at Φ=1.00): 1.00; HMRS(C₅₈H₆₈N₄O₄): calc. m/z: 884.524; found m/z: 884.522; Δ=−0.002.

### EXAMPLE 7

2,10-Bis[1-(1-methylethyl)-2-methylpropyl]-5-methyl-6-phenyl[1,3]-diazepino[4′,5′,6′:4,5]phenanthro[2,1,10-def::7,8,9-d′e′f′]diisoquinoline-1,3,9,11(2H,5H,9H,11H)-tetraone (4b). 2,10-Bis[1-(1-methylethyl)-2-methylpropyl]-6-phenyl[1,3]diazepino[4′,5′,6′:4,5]phenanthro[2,1,10-def::7,8,9-d′e′f′]diisoquinoline-1,3,9,11(2H,5H,9H,11H)-tetraone (2b) according to Example 4 (500 mg, 0.711 mmol) was converted and worked up analogously to Example 6, yield 450 mg (88%) of metallically shiny, violet colorant. M.p. >300° C.; R_{f} (silica gel, CHCl₃)=0.22; HMRS(C₄₆H₄₄N₄O₄): calc. m/z: 716.335; found m/z: 716.335, Δ=0.000 mmu.

### EXAMPLE 8

8,15-Bis(1-hexylheptyl)phenanthra[2,1,10-def:7,8,9-d′e′f′]-2,5-diphenyl-1,6,10,15-tetrahydroimidizo[4,5-h:4′,5′-h′]diisoquinoline-7,9,14,16-tetraone. N,N′-Bis(1-hexylheptyl)perylene-3,4:9,10-tetracarboximide ((1a), 1.00 g, 1.32 mmol) and NaNH₂ (1.00 g, 25.6 mmol) were suspended in benzonitrile (250 mL), heated to 165° C. (blue color), cooled again after 3 h, extracted by shaking with a 1:1 mixture of 2 N aqueous HCl and CHCl₃ (300 mL), dried over magnesium sulfate, concentrated with a rotary evaporator at 12 mbar and then under fine vacuum to remove benzonitrile, taken up in CHCl₃, filtered and purified by column chromatography using silica gel with chloroform as the eluent. 1^{st} green fraction: 2,9-bis(1-hexylheptyl)bis[1,3]diazepino[4′,5′,6′:1,12;4″,5″,6″:6,7]perylo[3,4-cd:9,10-c′d′]dipyridine-1,3,9,11 (2H,5H, 10H,13H)-tetraone. Yield 40 mg (4%) of black dye, m.p.: >250° C. R_{f} (silica gel, chloroform)=0.88. 2^{nd} fraction: 2,10-bis(1-hexylheptyl)-6-phenyl[1,3]diazepino[4′,5′,6′:4,5]phenanthro[2,1,10-def::7,8,9-d′e′f′]diisoquinoline-1,3,9,11 (2H, 5H, 9H,11H)-tetraone. Yield 409 mg (36%) of metallically shiny, violet dye of the formula (8). M.p. >250° C. R_{f} (silica gel, chloroform)=0.85. 3^{rd} fraction: 8,15-bis(1-hexylheptyl)phenanthra[2,1,10-def:7,8,9-d′e′f′]-2,5-diphenyl-1,6,10,15-tetrahydroimidizo[4,5-h:4′,5′-h′]diisoquinoline-7,9,14,16-tetraone. Yield 180 mg (18%) of green-black dye, m.p. >300° C. R_{f} (silica gel, chloroform)=0.23. IR (ATR): \tilde{v}=3387.6 m, 2952.9 m, 2920.8 s, 2853.6 m, 1685.1 m, 1639.5 s, 1593.9 s, 1582.7 s, 1545.9 w, 1486.1 w, 1456.3 w, 1429.2 w, 1402.4 w, 1381.5 w, 1349.0 m, 1325.2 m, 1304.9 m, 1290.2 m, 1242.9 s, 1173.7 m, 1127.5 w, 1026.1 w, 974.3 w, 879.7 w, 839.5 w, 810.7 m, 774.0 w, 753.0 w, 728.1 w, 701.4 w, 684.8, 624.9 w cm⁻¹. ¹H NMR (600 MHz, CDCl₃, 25° C.): δ=0.83 (t, 12H, ³J=7.0 Hz, CH₃), 1.24-1.42 (m, 32H, CH₂), 1.93-2.02 (m, 4H, β-CH₂), 2.28-2.39 (m, 4H, β-CH₂), 5.26-5.33 (m, 2H, CH—N), 7.49-7.54 (m, 4H, CH_{aryl}), 7.65-7.69 (m, 2H, CH_{aryl}), 8.20-8.80 (m, 4H, CH_{aryl}), 8.69-8.80 (m, 4H, H_{perylene}), 18.13 ppm (s, 1H, N—H). ¹³C NMR (150 MHz, CDCl₃, 25° C.): δ=14.1, 22.6, 27.2, 29.3, 29.4, 31.8, 32.5, 54.6, 54.9, 121.8, 124.4, 127.3, 128.6, 129.3, 132.2, 139.3, 163.9, 164.9 ppm. UV/Vis (CHCl₃) λₘₐₓ (Eᵣₑₗ)=390.0 (0.15), 435.0 (0.23), 486.0 (0.11), 524.0 (0.12), 555.0 (0.17), 598.0 (0.52), 651.0 (1.00). Fluorescence (CHCl₃): λₘₐₓ (Iᵣₑₗ)=670.0 (1.00), 735.1 (0.33) nm. Fluorescence quantum yield (CHCl₃, λ_{exc}=598 nm, E_{589 nm}=0.0249 cm⁻¹, reference: (1a) at 1.00): 0.80. MS (DEI⁺/70 eV): m/z (%)=989.5 (9), 988.5 (37) [M⁺+2H], 987.5 (82) [M⁺+H], 986.5 (100) [M⁺], 805.4 (13), 624.1 (31), 623.1 (65), 622.1 (32), 594.1 (23). C₆₄H₇₀N₆O₄ (987.3): calc. C, 77.86; H, 7.15; N, 8.51. found C, 77.83; H, 7.08; N, 8.47.

### EXAMPLE 9

2,10-Bis(1-hexylheptyl)-6-(2-bromophenyl)[1,3]diazepino[4′,5′,6′:4,5]-phenanthro[2,1,10-def::7,8,9-d′e′f′]diisoquinoline-1,3,9,11 (2H,5H,9H, 11H)-tetraone. N,N′-Bis(1-hexylheptyl)perylene-3,4:9,10-tetracarboximide (150 mg, 0.199 mmol) and NaNH₂ (150 mg, 3.85 mmol) are suspended in o-bromobenzonitrile (10 g) and converted and worked up analogously to 2,10-bis(1-hexylheptyl)-6-phenyl[1,3]diazepino[4′,5′,6′:4,5]phenanthro[2,1,10-def::7,8,9-d′e′f′]diisoquinoline-1,3,9,11 (2H, 5H, 9H,11H)-tetraone, and purified by column chromatography (silica gel, chloroform and isohexane (3:1)). Yield 91 mg (48.4%) of violet dye of the formula (8), m.p. >250° C. R_{f} (silica gel, chloroform)=0.80. ¹H NMR (300 MHz, CDCl₃, 25° C.): δ=0.80-0.93 (m, 12H, CH₃), 1.18-1.48 (m, 32H, CH₂), 1.83-2.02 (m, 4H, β-CH₂), 2.20-2.43 (m, 4H, β-CH₂), 5.15-5.35 (m, 2H, CH—N), 7.52 (t, 1H, ³J=7.6 Hz, CH_{aryl}), 7.69 (t, 1H, ³J=7.6 Hz, CH_{aryl}), 7.87 (d, 1H, ³J=7.6 Hz, CH_{aryl}), 8.60-8.88 (m, 6H, CH_{perylene}), 10.79 (d, 1H, ³J=7.8 Hz, CH_{perylene}) 12.41 ppm (s, 1H, N—H). UV/Vis (CHCl₃): λₘₐₓ (ε)=380.3 (9280), 397.1 (10070), 438.8 (13350), 461.2 (13030), 505.5 (16430), 542.9 (47470), 588.1 (86950). Fluorescence (CHCl₃): λₘₐₓ (Iᵣₑₗ)=601.5 (1.00), 654.1 (0.45). Fluorescence quantum yield (CHCl₃, λ_{exc}=542 nm, E_{542 nm}=0.0097 cm⁻¹, reference: (1a) at 1.00): 1.00. HRMS (C₅₇H₆₅⁷⁹BrN₄O₄): calc. m/z=948.4189; found m/z=948.4154, Δ=−0.0035. C₅₇H₆₅BrN₄O₄ (950.1): calc. C, 72.06; H, 6.90; N, 5.90. found C, 71.91; H, 6.76; N, 5.67.

### EXAMPLE 10

2,10-Bis(1-hexylheptyl)-6-(3-bromophenyl)[1,3]diazepino[4′,5′,6′:4,5]-phenanthro[2,1,10-def::7,8,9-d′e′f′]diisoquinoline-1,3,9,11(2H, 5H, 9H,11H)-tetraone. N,N′-Bis(1-hexylheptyl)perylene-3,4:9,10-tetracarboximide (200 mg, 0.265 mmol) and NaNH₂ (200 mg, 5.13 mmol) were suspended in m-bromobenzonitrile (10 g) and converted and worked up analogously to 2,10-bis(1-hexylheptyl)-6-phenyl[1,3]diazepino[4′,5′,6′:4,5]phenanthro[2,1,10-def::7,8,9-d′e′f′]diisoquinoline-1,3,9,11(2H,5H,9H,11H)-tetraone, and purified by column chromatography (silica gel, chloroform/isohexane 3:1). Yield 107 mg (42.4%) of violet dye of the formula (8), m.p. >250° C. R_{f} (silica gel, chloroform)=0.80. ¹H NMR (400 MHz, CDCl₃, 25° C.): δ=0.74-0.87 (m, 12H, CH₃), 1.13-1.45 (m, 32H, CH₂), 1.82-2.00 (m, 4H, β-CH₂), 2.20-2.37 (m, 4H, β-CH₂), 5.14-5.31 (m, 2H, CH—N), 7.53 (t, 1H, ³J=7.9 Hz, CH_{aryl}), 7.77 (ddd, 1H, ³J=8.0 Hz, ⁴J=1.8 Hz, ⁴J=0.8 Hz, CH_{aryl}), 8.25 (d, 1H, ³J=7.6 Hz, CH_{aryl}), 8.50-8.81 (m, 6H, CH_{perylene}) 1, 10.64 (d, 1H, ³J=8.2 Hz, CH_{perylene}), 11.48 ppm (s, 1H, N—H). ¹³C NMR (150 MHz, CDCl₃, 25° C.): δ=14.3, 22.8, 27.2, 29.5, 31.8, 32.0, 32.7, 54.9, 121.5, 123.0, 123.8, 123.8, 125.7, 125.8, 126.5, 126.8, 129.9, 129.3, 1230.4, 130.6, 131.0, 131.2, 132.6, 134.8, 135.2, 135.5, 139.0, 139.2, 143.7, 155.8, 164.0, 165.0 ppm. UV/Vis (CHCl₃): λₘₐₓ (Eᵣₑₗ)=378.6 (0.10), 399.1 (0.10), 437.2 (0.13), 461.4 (0.13), 504.6 (0.16), 542.7 (0.51), 587.4 (1.00). Fluorescence (CHCl₃): λₘₐₓ (Iᵣₑₗ)=595.8 (1.00), 648.8 (0.42). Fluorescence quantum yield (CHCl₃, λ_{exc}=542 nm, E_{542 nm}=0.0068 cm⁻¹, reference: (1a) at 1.00): 0.99. HRMS (C₅₇H₆₅⁷⁹BrN₄O₄): calc. m/z=948.4189; found m/z=948.4177, Δ=−0.0012.

### EXAMPLE 11

2,10-Bis(1-hexylheptyl)-6-(4-dimethylaminophenyl)[1,3]diazepino-[4′,5′,6′-d″,e″,f″]phenanthra[2,1,10-def:7,8,9-d′e′f′]-2,5,9,11-tetrahydro-diisoquinoline-1,3,9,11-tetraone. N,N′-Bis(1-hexylheptyl)perylene-3,4:9,10-tetracarboximide (200 mg, 0.26 mmol) and NaNH₂ (200 mg, 5.13 mmol) were suspended in 4-dimethylaminobenzonitrile (10 g) and converted and worked up analogously to 2,10-bis(1-hexylheptyl)-6-phenyl[1,3]diazepino[4′,5′,6′:4,5]-phenanthro[2,1,10-def::7,8,9-d′e′f′]diisoquinoline-1,3,9,11(2H, 5H,9H,11H)-tetraone, and purified by column chromatography (silica gel, CHCl₃). The dye of the formula (8) is dissolved in chloroform and precipitated with methanol. Yield 23 mg (12%) of metallically shiny, violet dye, m.p. >300° C. R_{f} (silica gel, chloroform)=0.70. IR (ATR): \tilde{v}=3416.2 m, 3096.0 w, 2952.8 s, 2921.7 s, 2853.8 s, 2364.3 w, 1691.1 m, 1639.8 s, 1626.6 s, 1607.3 s, 1590.8 s, 1493.2 m, 1470.3 m, 1434.2 m, 1413.5 m, 1365.8 m, 1343.8 s, 1306.2 m, 1255.9 s, 1219.9 m, 1196.3 m, 1121.0 m, 1103.5 m, 1058.3 m, 1038.2 w, 949.8 w, 871.3 w, 809.8 m, 751.4 w, 619.1 w, 581.6 w cm⁻¹. ¹H NMR (600 MHz, CDCl₃, 25° C.): δ=0.82-0.87 (m, 12H, CH₃), 1.25-1.46 (m, 32H, CH₂), 1.88-1.99 (m, 4H, β-CH₂), 2.25-2.35 (m, 4H, β-CH₂), 3.17 (s, 6H, NCH₃), 5.21-5.31 (m, 2H, CH—N), 6.87 (d, 2H, ³J=8.2 Hz, CH_{aryl}), 8.15 (d, 2H, ³J=8.2 Hz, CH_{aryl}), 8.46-8.73 (m, 5H, H_{perylene}), 10.65 (d, 1H, ³J=8.1 Hz), 11.25 ppm (s, 1H, N—H). ¹³C NMR (150 MHz, CDCl₃, 25° C.): δ=14.1, 14.1, 22.6, 27.0, 29.3, 31.8, 31.8, 32.5, 40.4, 54.6, 112.1, 120.8, 122.1, 123.4, 126.1, 126.6, 129.2, 129.3, 130.0, 130.8, 134.7, 134.9, 135.0, 144.8, 152.6, 161.8, 164.0, 165.0 ppm. UV/Vis (CHCl₃): λₘₐₓ (Eₘₐₓ)=396.0 (0.20), 513 (sh, 0.67), 544 (1.0), 594 (sh, 0.91), 616 nm (0.98). Fluorescence (CHCl₃): λₘₐₓ=740.2 nm. Fluorescence quantum yield (CHCl₃, λ_{exc}=465 nm, E_{465 nm}=0.0476 cm⁻¹, reference: (1a) at Φ=1.00): 0.72. HRMS (C₅₉H₇₁N₅O₄): calc. m/z:=913.550; found m/z=913.549, Δ=−0.0010.

### EXAMPLE 12

2,10-Bis(1-hexylheptyl)-6-(2,4-bismethoxyphenyl)[1,3]diazepino-[4′,5′,6′-d″,e″,f″]phenanthra[2,1,10-def:7,8,9-d′e′f′]-2,5,9,11-tetrahydro-diisoquinoline-1,3,9,11-tetraone. N,N′-Bis(1-hexylheptyl)perylene-3,4:9,10-tetracarboximide (200 mg, 0.265 mmol) and NaNH₂ (200 mg, 5.13 mmol) were dissolved in 2,4-bismethoxybenzonitrile (10 g) at 100° C. and converted and worked up analogously to 2,10-bis(1-hexylheptyl)-6-phenyl[1,3]diazepino[4′,5′,6′:4,5]-phenanthro[2,1,10-def::7,8,9-d′e′f′]diisoquinoline-1,3,9,11(2H,5H,9H,11H)-tetraone, and purified by column chromatography (silica gel, chloroform). Yield 80 mg (32.4%) of metallically shiny, violet dye of the formula (8). M.p. >300° C. R_{f} (silica gel, chloroform)=0.38. UV/Vis (CHCl₃): λₘₐₓ (Eᵣₑₗ)=396.9 (0.14), 464.3 (0.29), 491.4 (0.37), 560.3 (0.62), 603.5 (1.00). Fluorescence (CHCl₃): λₘₐₓ (Iᵣₑₗ)=634.2 (1.00), 684.6 (0.66). Fluorescence quantum yield (CHCl₃, λ_{exc}=560 nm, E_{560 nm}=0.01088 cm⁻¹, reference: (1a) at Φ=1.00): 0.96. ¹H NMR (400 MHz, CDCl₃, 25° C.): δ=0.77-0.87 (m, 12H, CH₃), 1.17-1.43 (m, 32H, CH₂), 1.81-1.98 (m, 4H, β-CH₂), 2.20-2.40 (m, 4H, β-CH₂), 3.96 (s, 3H, OCH₃), 4.15 (s, 3H, OCH₃), 5.15-5.30 (m, 2H, CH—N), 6.61 (d, 2H, ³J=1.8 Hz, CH_{aryl}), 6.80 (d, 2H, ³J=9.1 Hz, CH_{aryl}), 8.50-8.79 (m, 6H, H_{perylene}), 10.60-10.72 (br, 1H), 12.13-12.24 ppm (br, 1H, N—H). ¹³C NMR (100 MHz, CDCl₃, 25° C.): δ=14.3, 22.9, 27.3, 29.5, 32.0, 32.7, 54.8, 56.0, 56.5, 98.9, 106.8, 109.8, 121.2, 122.4, 123.6, 126.3, 126.9, 129.4, 130.2, 130.5, 133.2, 135.0, 135.4, 143.7, 159.9, 164.5, 165.4 ppm. HRMS (C₅₉H₇₀N₄O₆): calc. m/z=930.5295; found m/z=930.5289, Δ=−0.0006.

### EXAMPLE 13

2,10-Bis(1-hexylheptyl)-6-(3,4-bismethoxyphenyl)-[1,3]diazepino-[4′,5′,6′-d″,e″,f″]phenanthra[2,1,10-def:7,8,9-d′e′f′]-2,5,9,11-tetrahydro-diisoquinoline-1,3,9,11-tetraone. N,N′-Bis(1-hexylheptyl)perylene-3,4:9,10-tetracarboximide (0.240 g, 0.317 mmol) and NaNH₂ (0.200 g, 5.13 mmol) were dissolved in 2,4-bismethoxybenzonitrile (10 g) at 100° C. and converted (reaction time 4.5 h) and worked up analogously to 2,10-bis(1-hexylheptyl)-6-phenyl[1,3]diazepino[4′,5′,6′:4,5]phenanthro[2,1,10-def::7,8,9-d′e′f′]diisoquinoline-1,3,9,11(2H,5H,9H,11H)-tetraone, and purified by column chromatography (silica gel, chloroform). Yield: 118 mg (39.9%) of metallically shiny, violet dye of the formula (8), m.p. >300° C. R_{f} (silica gel, chloroform)=0.38. ¹H NMR (600 MHz, CDCl₃, 25° C.): δ=0.78-0.85 (m, 12H, CH₃), 1.17-1.44 (m, 32H, CH₂), 1.83-1.98 (m, 4H, β-CH₂), 2.21-2.34 (m, 4H, β-CH₂), 4.04 (s, 3H, OCH₃), 4.11 (s, 3H, OCH₃), 5.15-5.28 (m, 2H, CH—N), 7.07 (d, 2H, ³J=8.4 Hz, CH_{aryl}), 7.78-7.87 (m, 2 H, CH_{aryl}), 8.44-8.75 (m, 5H, H_{perylene}), 10.63-10.68 (br, 1H), 11.35 ppm (s, 1H, N—H). ¹³C NMR (100 MHz, CDCl₃, 25° C.): δ=14.3, 14.3, 22.8, 22.8, 27.2, 27.3, 27.4, 32.5, 32.7, 32.8, 54.8, 54.9, 56.5, 56.5, 103.4, 104.2, 111.6, 121.0, 121.3, 121.5, 122.7, 123.6, 126.6, 126.8, 129.4, 130.6, 144.3, 150.0, 153.0, 157.7, 164.1, 165.3, 166.0 ppm. UV/Vis (CHCl₃): λₘₐₓ (Eᵣₑₗ)=500.9 (0.29), 549.4 (0.58), 593.7 (1.00). Fluorescence (CHCl₃): λₘₐₓ (Iᵣₑₗ)=614.8 (1.00), 665.7 (0.61). Fluorescence quantum yield (CHCl₃, λ_{exc}=549 nm, E_{549 nm}=0.008146 cm⁻¹, reference: (1a) at Φ=1.00): 1.00. HRMS (C₅₉H₇₀N₄O₆): calc. m/z=930.5295; found m/z=930.5284, Δ=−0.0011. C₅₉H₇₀N₄O₆ (931.2): calc. C, 76.10; H, 7.58; N, 6.02. found C, 75.66; H, 7.38; N, 5.90.

### EXAMPLE 14

Dinitrogen tetroxide in dichloromethane: Solid lead(II) nitrate (100 g, 302 mmol) is heated strongly with a Bunsen burner in a round-bottomed flask and the nitrous gases which form are passed into a reservoir flask containing dichloromethane (1000 mL) until the dichloromethane solution is saturated. This reagent is used for the alternative synthesis route according to the examples which follow.

### EXAMPLE 15

1-Nitro-N,N′-bis(1-hexylheptyl)perylene-3,4:9,10-bis(dicarboximide). N,N′-Bis(1-hexylheptyl)perylene-3,4:9,10-tetracarboximide (1a, 3.00 g, 3.97 mmol) was initially charged in dichloromethane (200 mL), admixed with methanesulfonic acid (2 mL, 30.8 mmol) (catalyst in excess), admixed dropwise with a sufficient amount of a saturated solution of dinitrogen tetroxide in dichloromethane (according to Example 14) at room temperature while stirring until monitoring by means of thin-layer chromatography (silica gel, chloroform) showed complete conversion (color change of the solution to dark red), washed with distilled water (100 mL), dried over magnesium sulfate, concentrated with a rotary evaporator, dissolved in a little chloroform and purified by column chromatography (silica gel, dichloromethane). Yield 2.41 g (76%) of dark red, metallically shiny solid, m.p. 120° C. R_{f} (silica gel/CHCl₃): 0.80. IR (ATR): \tilde{v}=3048 w, 2955 s, 2927 s, 2857 s, 1703 s, 1661 s, 1596 s, 1537 s, 1457 m, 1427 m, 1405 s, 1337 s, 1251 s, 1209 w, 1179 m, 1112 w, 973 w, 920 w, 855 w, 812 m, 746 cm⁻¹ m. ¹H NMR (300 MHz, CDCl₃, 25° C.): δ=0.83 (t, 6H, ³J=7.0 Hz, CH₃), 1.21-1.38 (m, 16H, CH₂), 1.85-1.91 (m, 2H, β-CH₂), 2.22-2.28 (m, 2H, β-CH₂), 5.10-5.20 (m, 2H, α-CH₂), 8.25 (d, ³J=8.1 Hz, 1H, H_{perylene}), 8.59 (d, ³J=8.1 Hz, 1H, H_{perylene}), 8.71-8.77 (m, 5H, H_{perylene}). UV/Vis (CHCl₃): λₘₐₓ (Eᵣₑₗ)=490 (0.67), 523 nm (1.0).

### EXAMPLE 16

1-Amino-N,N′-bis(1-hexylheptyl)perylene-3,4:9,10-bis(dicarboximide).

1-Nitro-N,N′-bis(1-hexylheptyl)perylene-3,4:9,10-bis(dicarboximide) according to Example 15 (2.41 g, 3.01 mmol) was dissolved in THF (100 mL) (dark red solution), admixed with iron powder (350 mg, 6.27 mmol) and concentrated hydrochloric acid (11 mL), heated under reflux while stirring for 30 minutes (after 10 minutes a color change from dark red to dark blue), allowed to cool, precipitated with distilled water (500 mL), filtered off with suction, dissolved in a little chloroform, purified by column chromatography (silica gel, chloroform) and concentrated by evaporation. Yield 2.08 g (72.1%) of very fine, amorphous solid, m.p. 92-94° C. R_{f} (silica gel/CHCl₃): 0.30. IR (ATR): \tilde{v}=3350 m, 3242 m, 3047 w, 2954 s, 2926 s, 2856 s, 1694 s, 1653 s, 1616 m, 1590 s, 1573 m, 1510 m, 1463 m, 1429 s, 1397 m, 1373 m, 1339 s, 1311 m, 1269 m, 1252 m, 1178 m, 1122 w, 1084 m, 1062 w, 979 w, 846 w, 809 m, 750 w, 725 cm⁻¹ w. ¹H NMR (300 MHz, CDCl₃, 25° C.): δ=0.83 (t, 6H, ³J=7.0 Hz, CH₃), 1.21-1.38 (m, 16H, CH₂), 1.85-1.91 (m, 2H, β-CH₂), 2.22-2.28 (m, 2 H, β-CH₂), 5.10-5.20 (m, 4H, α-CH₂, NH₂), 8.17 (s, 1H, H_{perylene}), 8.49-8.55 (m, 3H, H_{perylene}), 8.64 (s, 2H, H_{perylene}), 8.87 ppm (d, ³J=8.1 Hz, 1H, H_{perylene}). UV/Vis (CHCl₃): λₘₐₓ (Eᵣₑₗ)=420 (0.32), 571 nm (1.00). Fluorescence (CHCl₃): λₘₐₓ=684 nm.

### EXAMPLE 17

1-Benzamidyl-N,N′-bis(1-hexylheptyl)perylene-3,4:9,10-tetracarboximide. 1-Amino-N,N′-bis(1-hexylheptyl)perylene-3,4:9,10-bis(dicarboximide) according to Example 16 (240 mg, 0.31 mmol) was dissolved in dioxane (60 mL), admixed dropwise with benzoyl chloride (1.00 g, 7.10 mmol) in dioxane (10 mL), stirred under reflux at 100° C. for 7 h (after 2 h, the same amount of benzoyl chloride again is added (monitoring of the end of the reaction by means of TLC)), filtered, purified by column chromatography (silica gel, chloroform, red first runnings composed of the starting material, pink main fraction), concentrated with a rotary evaporator, taken up in a little chloroform and precipitated with methanol, filtered off with suction and dried at 110° C. in a drying cabinet. Yield 125 mg (46%) of pink dye of the formula (7), m.p. >300° C. R_{f} (silica gel, CH₂Cl₂)=0.53. IR (ATR): \tilde{v}=3212.5 w, 2952.1 m, 2921.4 s, 2853.3 m, 1697.0 s, 1654.9 s, 1591.4 m, 1527.1 w, 1503.1 w, 1482.2 w, 1466.4 m, 1455.4 m, 1407.9 m, 1362.2 w, 1326.9 s, 1267.5 m, 1246.2 m, 1175.6 m, 1106.9 w, 1025.0 w, 971.7 w, 940.7 w, 896.9 w, 845.5 w, 809.2 m, 745.5 w, 702.2 w, 686.8 w, 614.9 w cm⁻¹. ¹H NMR (600 MHz, CDCl₃, 25° C.): δ=0.82 (t, ³J_{H,H}=7.0 Hz, 12H, CH₃), 1.16-1.35 (m, 33H, CH₂ and NH), 1.79-1.85 (m, 4H, β-CH₂), 2.15-2.25 (m, 4H, β-CH₂), 5.10-5.19 (m, 2H, α-CH), 7.59 (t, ³J=7.7 Hz, 2H, phenyl), 8.04 (d, ³J=7.3 Hz, 1H, phenyl), 8.45 (s, 2H, phenyl), 8.52 (d, ³J=7.9 Hz, 1H, H_{perylene}), 8.60-8.64 (m, 2H, phenyl), 8.79 (s, 1H, CH_{perylene}), 8.97-8.99 ppm (m, 2H, CH_{perylene}). ¹³C NMR (150 MHz, CDCl₃, 25° C.): δ=14.0, 22.6, 26.9, 29.2, 31.7, 31.8, 32.3, 54.8, 54.9, 122.5, 123.7, 125.6, 127.0, 127.5, 128.0, 129.2, 132.9, 133.2, 134.3, 134.3, 135.2, 135.2, 165.8 ppm. UV/Vis (CHCl₃): λₘₐₓ (Eᵣₑₗ)=274 (0.51), 313 (0.29), 380 (0.12), 419 (0.12), 498 (0.71), 531 nm (1.00). Fluorescence (CHCl₃): λₘₐₓ=599 nm. Fluorescence quantum yield (CHCl₃, λ_{exc}=500 nm, E_{500 nm}=0.01835 cm⁻¹, reference: (1a) at 1.00): 0.813. HRMS (C₅₇H₆₇N₃O₅): calc. m/z=873.508; found m/z=873.508, Δ=0.0000. C₅₇H₆₇N₃O₅ (874.16): calc. C, 78.32; H, 7.73; N, 4.81. found C, 77.97; H, 7.43; N, 4.73.

### EXAMPLE 18

2,10-Bis(1-hexylheptyl)-6-phenyl[1,3]diazepino[4′,5′,6′-d″,e″,f″]phenanthra[2,1,10-def:7,8,9-d′e′f′]-2,5,9,11-tetrahydrodiisoquinoline-1,3,9,11-tetraone. 1-Benzamidyl-N,N′-bis(1-hexylheptyl)perylene-3,4:9,10-tetracarboxylic acid 3,4-anhydride 9,10-imide (100 mg, 0.110 mmol) and NaNH₂ (100 mg, 2.56 mmol) were suspended in benzonitrile (250 mL), heated to 165° C. (blue color), cooled again after 3 h, extracted by shaking with a 1:1 mixture of HCl solution (2 N) and CHCl₃ (300 mL), freed of the CHCl₃, freed of the benzonitrile by distillation under reduced pressure, taken up in a little CHCl₃, filtered and purified by column chromatography (silica gel, CHCl₃). Yield 60 mg (57%) of metallically shiny, violet dye.

### EXAMPLE 19

Exhaustive nitration of N,N′-Bis(1-hexylheptyl)perylene-3,4:9,10-tetracarboximide-N,N′-bis(1-hexylheptyl)-1,6-dinitroperylene-3,4:9,10-tetracarboximide and N,N′-bis(1-hexylheptyl)-1,7-dinitroperylene-3,4:9,10-tetracarboximide. N,N′-Bis(1-hexylheptyl)perylene-3,4:9,10-tetracarboximide ((1a), 3.19 g, 4.23 mmol) was dissolved in a little dichloromethane, admixed with methanesulfonic acid (2 mL) and dinitrogen tetroxide solution, stirred while irradiating with light (80 W tungsten filament lamp) at room temperature for 6 h, stopped by admixing with distilled water (100 ml), extracted repeatedly with chloroform (100 ml each time), dried over magnesium sulfate, concentrated and chromatographed (silica gel, chloroform). This affords a mixture of N,N′-bis(1-hexylheptyl)-1,6-dinitroperylene-3,4:9,10-tetracarboximide and N,N′-bis(1-hexylheptyl)-1,7-dinitroperylene-3,4:9,10-tetracarboximide as a deep red solid. A separation of these two isomers did not succeed owing to identical R_{f} values. Yield 2.72 g (76.1%). R_{f} (silica gel/CHCl₃): 0.7. IR (ATR): \tilde{v}=3047 w, 2978 m, 2927 s, 2857 m, 1705 s, 1664 s, 1599 s, 1542 s, 1427 w, 1407 m, 1335 s, 1251 m, 812 m, 743 cm⁻¹ w. ¹H NMR (300 MHz, CDCl₃, 25° C.): δ=0.83 (t, 6H, ³J=7.0 Hz, CH₃), 1.21-1.38 (m, 16H, CH₂), 1.85-1.91 (m, 2H, β-CH₂), 2.22-2.28 (m, 2H, β-CH₂), 5.10-5.20 (m, 2H, α-CH₂), 8.31 (d, ³J=8.0 Hz, 1H, CH_{perylene}), 8.33 (d, ³J=8.0 Hz, 1H, CH_{perylene}), 8.66-8.70 (m, 2H, CH_{perylene}), 8.83 (s, 2H, H_{perylene}). UV/Vis (CHCl₃): λₘₐₓ (Eᵣₑₗ)=450 (0.26), 491 (0.73), 520 nm (1.00).

### EXAMPLE 20

N,N′-bis(1-hexylheptyl)-1,6-diaminoperylene-3,4:9,10-tetracarboximide and N,N′-bis(1-hexylheptyl)-1,7-diaminoperylene-3,4:9,10-tetracarboximide isomer mixture. The N,N′-bis(1-hexylheptyl)-1,6-dinitroperylene-3,4:9,10-tetracarboximide and N,N′-bis(1-hexylheptyl)-1,7-dinitroperylene-3,4:9,10-tetracarboximide isomer mixture from the nitration of N,N′-bis(1-hexylheptyl)perylene-3,4:9,10-tetracarboximide (1.00 g, 1.19 mmol) was dissolved in boiling ethanol (150 mL) under reflux, admixed with iron powder (500 mg, 8.93 mmol) and hydrochloric acid (conc., 5.00 mL), stirred for 30 min, stopped by adding distilled water (500 mL), stirred at room temperature for a further hour, filtered and chromatographed (silica gel, dichloromethane). Yield 814 mg (87.6%), m.p. 146-148° C. R_{f} (silica gel/CHCl₃):0.20. IR (ATR): \tilde{v}=3441 s br., 3360 s, 3250 m, 2954 m, 2926 s, 2857 m, 1691 s, 1646 s, 1588 s, 1552 w, 1530 w, 1515 w, 1455 m, 1422 m, 1393 m, 1339 s, 1272 m, 1246 w, 1185 w, 1107 w, 984 w, 873 m, 806 m, 778 w, 755 m, 723 w, 574 cm⁻¹ w. UV/Vis (CHCl₃): λₘₐₓ (Eᵣₑₗ)=384 nm (0.19), 402 (0.25), 432 (0.16), 506 sh (0.32), 581 sh (0.90), 611 (1.0).

### EXAMPLE 21

1,6-Bis(benzamidyl)-N,N′-bis(1-hexylheptyl)perylene-3,4:9,10-tetra-carboximide and 1,7-bis(benzamidyl)-N,N′-bis(1-hexylheptyl)perylene-3,4:9,10-tetracarboximide. A solution of the N,N′-bis(1-hexylheptyl)-1,6-diaminoperylene-3,4:9,10-tetracarboximide and N,N′-bis(1-hexylheptyl)-1,7-diaminoperylene-3,4:9,10-tetracarboximide isomer mixture (1.00 g, 1.27 mmol) in 1,4-dioxane (100 mL) was admixed dropwise with benzoyl chloride (2.00 g, 14.2 mmol) in 1,4-dioxane (10 mL), stirred under reflux at 100° C. for a total of 7 h (every 2 h, benzoyl chloride (2.00 g) was added and the end of the reaction is monitored by means of TLC), concentrated, freed of benzoyl chloride by distillative removal, purified by column chromatography (silica gel, chloroform, pink product after some preliminary fractions), concentrated with a rotary evaporator and freed of a colorless liquid under fine vacuum at 200° C. Yield 450 mg (35%) of metallically shiny dye, m.p. >300° C.

R_{f} (silica gel, chloroform)=0.05. IR (ATR): \tilde{v}=3222.2 m, 2952.2 m, 2922.0 s, 2854.0 m, 1699.4 s, 1654.9 s, 1592.0 s, 1502.4 w, 1478.9 w, 1455.4 w, 1409.3 w, 1322.4 s, 1267.3 m, 1243.3 m, 1180.4 w, 1108.0 w, 1026.1 w, 895.7 w, 862.1 m, 809.1 w, 749.4 m, 704.0 m, 585.4 w cm⁻¹. ¹H NMR (600 MHz, CDCl₃, 25° C.): δ=0.82-0.86 (m, 12H, CH₃), 1.11-1.26 (m, 32H, CH₂), 1.80-2.10 (m, 8H, β-CH₂), 4.90-5.02 (m, 2H, CH—N), 7.48-7.66 (m, 5H, CH_{aryl}), 7.87-7.92 (m, 3H, CH_{aryl}), 8.18-8.22 (m, 2H, CH_{aryl}), 8.58-8.80 ppm (m, 6H, H_{pery}). ¹³C NMR (150 MHz, CDCl₃, 25° C.): δ=14.1, 14.1, 14.1, 22.6, 22.6, 22.6, 27.0, 27.0, 29.3, 29.3, 29.4, 31.7, 31.7, 31.7, 32.1, 32.3, 54.8, 55.2, 124.8, 125.1, 126.1, 126.1, 127.4, 127.7, 127.9, 128.3, 128.3, 128.3, 128.3, 128.8, 128.8, 128.8, 128.8, 129.2, 129.4, 130.3, 131.0, 132.7, 133.7, 135.4, 162.4, 163.0, 164.1, 165.9 ppm. UV/Vis (CHCl₃): λₘₐₓ (Eᵣₑₗ)=387 (0.36), 544 nm (1.0). Fluorescence (CHCl₃): λₘₐₓ=615 nm. Fluorescence quantum yield (CHCl₃, λ_{exc}=521 nm, E₅₂₁ nm=0.03251 cm⁻¹, reference: (1a) at 1.00): 0.53. MS (EI): m/z (%)=995.6 [M⁺+2 H] (6), 994.6 [M⁺+H] (16), 993.6 [M⁺] (22), 874.2 (9).

### EXAMPLE 22

1,6-Bis(benzamidyl)-N,N′-bis(1-hexylheptyl)perylene-3,4:9,10-tetracarboximide and 1,7-bis(benzamidyl)-N,N′-bis(1-hexylheptyl)perylene-3,4:9,10-tetracarboximide (100 mg, 0.10 mmol) as an isomer mixture and NaNH₂ (100 mg, 2.56 mmol) were suspended in benzonitrile (250 mL), heated to 165° C. (blue color), cooled after 3 h and extracted by shaking with a 1:1 mixture of aqueous HCl (2 N) and CHCl₃ (300 mL). The organic phase was concentrated by evaporation, freed of the benzonitrile under fine vacuum, taken up in a little CHCl₃, filtered and purified by column chromatography (silica gel, CHCl₃). The eluted dye was taken up in a little dichloromethane and precipitated with methanol. Yield 37 mg (35%) of black dye of the formula (9) and/or (10).

### EXAMPLE 23

2,10-Bis(1-hexylheptyl)-(N-benzyl)-6-phenyl[1,3]diazepino[4′,5′,6′:4,5]-phenanthro[2,1,10-def::7,8,9-d′e′f′]diisoquinoline-1,3,9,11(2H,5H,9H,11H)-tetraone. 2,10-Bis(1-hexylheptyl)-6-phenyl[1,3]diazepino[4′,5′,6′:4,5]phenanthro[2,1,10-def::7,8,9-d′e′f′]diisoquinoline-1,3,9,11(2H,5H,9H,11H)-tetraone (85 mg, 0.098 mmol) was dissolved in DMF (5 mL), heated to 100° C., admixed with anhydrous potassium carbonate (100 mg, 0.724 mmol) (blue color of the previously pink solution), admixed dropwise with benzyl bromide (400 mg, 2.34 mmol) (violet color), stirred at 100° C. for 3 h, stopped by adding 2 N HCl solution, filtered off with suction, dried at 110° C. for 16 h and purified by column chromatography (silica gel, chloroform/isohexane 3:1). Yield 26 mg (27.7%) of dark violet solid of the formula (7), m.p. >250° C. R_{f}(silica gel/chloroform)=0.81. IR (ATR): \tilde{v}=3061.3 vw, 2953.6 m, 2922.4 vs, 2854.4 s, 2359.2 w, 2341.1 w, 1685.5 vs, 1641.8 vs, 1590.8 s, 1573.8 m, 1529.9 w, 1483.8 w, 1455.2 w, 1424.0 m, 1406.6 vw, 1378.0 w, 1357.6 w, 1330.2 vs, 1251.6 m, 1222.4 w, 1180.7 w, 1108.5 w, 1075.2 w, 1027.9 w, 873.3 w, 845.7 w, 809.0 m, 774.1 w, 751.3 w, 720.6 w, 699.0 m, 673.7 w cm⁻¹. ¹H NMR (150 MHz, CDCl₃, 25° C.): δ=0.83 (t, ³J=6.9 Hz, 12H, CH₃), 1.18-1.40 (m, 32H, CH₂), 1.85-1.94 (m, 4H, CH₂), 2.15 (s, 2H, CH₂), 2.23-2.32 (m, 2H, CH₂), 5.06-5.24 (m, 2H, CH—N), 6.19 (s, 1H, N—CH₂), 6.25 (s, 1H, N—CH₂), 6.54-6.64 (br, 2H, CH_{aromat,benzyl}), 6.95-7.04 (br, 2H, CH_{aromat,benzyl}), 7.67 (s, 3H, CH_{phenyl}), 8.06 (s, 2H, CH_{phenyl}), 8.58-8.82 (m, 5H, CH_{perylene}), 10.84 ppm (s, 1H, CH_{perylene}). ¹³C NMR (150 MHz, CDCl₃, 25° C.): δ=14.3, 14.3, 22.8, 22.8, 27.2, 27.3, 29.5, 29.6, 32.1, 32.0, 32.7, 54.9, 54.9, 55.1, 75.8, 121.5, 122.8, 125.6, 126.1, 126.5, 127.0, 128.8, 129.3, 129.4, 129.9, 130.6, 131.5, 135.0, 145.0, 164.0, 165.1 ppm. UV/Vis (CHCl₃): λₘₐₓ (Eᵣₑₗ)=395 (0.11), 435 (0.11), 500 (0.17), 537 (0.52), 582 (1.00). Fluorescence (CHCl₃): λₘₐₓ (Iᵣₑₗ)=593 (1.00), 647 nm (0.44). Fluorescence quantum yield (λ_{exc}=490 nm, E_{490 nm}=0.13671/1 cm, CHCl₃, reference (1a) at Φ=1.00): Φ=1.00. MS (DEI⁺, 70 eV): m/z (%): 964.4 (6.3), 963.3 (24.9), 962.3 (69.8), 961.3 (100.0) [M⁺], 871.1 [M⁺-benzyl], 780.7 (5.0), 779.7 (11.4), 778.7 (11.3) [M⁺-C₁₃H₂₆], 598.2 (6.1), 597.2 (18.8), 596.2 (28.6), 595.2 (14.9) [M⁺−2C₁₃H₂₆].

### EXAMPLE 24

2,10-Bis(1-hexylheptyl)-(N-4-tert-butylbenzyl)-6-phenyl[1,3]diazepino-[4′,5′,6′:4,5]phenanthro[2,1,10-def::7,8,9-d′e′f′]diisoquinoline-1,3,9,11(2H,5H,9H,-11H)-tetraone. 2,10-Bis(1-hexylheptyl)-6-phenyl[1,3]diazepino[4′,5′,6′:4,5]-phenanthro[2,1,10-def::7,8,9-d′e′f′]diisoquinoline-1,3,9,11(2H, 5H, 9H,11H)-tetraone (85 mg, 0.098 mmol) was reacted in DMF (5 mL) with potassium carbonate (100 mg, 0.724 mmol) and 4-tert-butylbenzyl bromide (400 mg, 1.76 mmol) as for 2,10-bis(1-hexylheptyl)-(N-benzyl)-6-phenyl[1,3]diazepino[4′,5′,6′:4,5]-phenanthro[2,1,10-def::7,8,9-d′e′f′]diisoquinoline-1,3,9,11(2H,5H,9H,11H)-tetraone, and worked up and purified by column chromatography (silica gel, chloroform/iso-hexane 2:1). Yield 64 mg (64.5%) of black-violet solid of the formula (7), m.p. >250° C. R_{f} (silica gel/chloroform:isohexane 2:1): 0.40. IR (ATR): \tilde{v}=3058.1 vw, 2953.6 m, 2922.6 vs, 2854.5 s, 2360.5 w, 2340.9 w, 1687.1 vs, 1643.3 vs, 1591.3 s, 1573.9 m, 1529.7 w, 1483.8 w, 1463.0 w, 1424.0 m, 1407.4 vw, 1377.6 w, 1357.6 w, 1330.1 vs, 1251.9 m, 1223.0 w, 1179.4 w, 1107.2 w, 1025.1 w, 929.4 vw, 873.3 w, 844.9 w, 809.8 m, 774.1 w, 750.7 w, 719.5 w, 700.8 m, 673.0 w cm⁻¹. ¹H NMR (600 MHz, CDCl₃, 25° C.): δ=0.81 (t, ³J=7.0 Hz, 12H, CH₃), 1.07 (s, 9H, tert-butyl-CH₃), 1.16-1.40 (m, 32H, CH₂), 1.83-1.97 (m, 4H, CH₂), 2.12-2.31 (m, 4H, CH₂), 5.13-5.23 (m, 2H, CH—N), 6.14 (s, 1H, N—CH₂), 6.19 (s, 1H, N—CH₂), 6.46-6.61 (br, 2H, CH_{aromat,benzyl}), 7.00 (d, ³J=8.3 Hz, 2H, CH_{aromat,benzyl}), 7.65 (s, 3H, CH_{phenyl}), 8.06 (s, 2H, CH_{phenyl}), 8.47-8.79 (m, 5H, CH_{perylene}) 10.80 ppm (s, 1H, CH_{perylene}). ¹³C NMR (150 MHz, CDCl₃, 25° C.): δ=14.3, 14.3, 22.8, 22.9, 27.2, 27.5, 29.5, 31.3, 32.0, 32.1, 32.7, 34.6, 54.9, 76.6, 121.3, 122.7, 123.8, 125.5, 125.7, 126.1, 126.7, 126.9, 129.2, 129.5, 130.0, 130.7, 131.4, 134.8, 139.9, 145.0, 164.0, 165.1 ppm. UV/Vis (CHCl₃): λₘₐₓ (Eᵣₑₗ)=395 (0.11), 436 (0.12), 501 (0.17), 538 (0.52), 582 (1.00). Fluorescence (CHCl₃): λₘₐₓ (Iᵣₑₗ)=595 (1.00), 648 nm (0.45). Fluorescence quantum yield (λ_{exc}=490 nm, E_{490 nm}=0.013671/1 cm, CHCl₃, reference (2a) at Φ=1.00): 1.00. MS (DEI⁺, 70 eV): m/z (%): 1020.6 (7.5), 1019.6 (28.7), 1018.6 (73.0), 1017.6 (100.0) [M⁺], 872.1 (9.0), 871.1 (15.4) [M⁺-benzyl], 835.9 (8.6), 834.9 (9.0) [M⁺-C₁₃H₂₆], 653.4 (6.7), 652.4 (11.2) [M⁺−2 C₁₃H₂₆], 651.4 (6.6). C₆₈H₈₀N₄O₄ (1017.39): calc. C, 80.28; H, 7.93; N, 5.51. found C, 79.90; H, 7.38; N, 5.39.

### EXAMPLE 25

Bichromophore composed of monoadduct and perylenebisimide. 2,10-Bis(1-hexylheptyl)-6-phenyl[1,3]diazepino[4′,5′,6′:4,5]phenanthro[2,1,10-def::7,8,9-d′d′f′]diisoquinoline-1,3,9,11(2H,5H,9H,11H)-tetraone (100 mg, 0.115 mmol) was reacted in DMF (5 mL) with potassium carbonate (100 mg, 0.724 mmol) and perylenebenzyl bromide (400 mg, 0.529 mmol) as for 2,10-bis(1-hexylheptyl)-(N-benzyl)-6-phenyl[1,3]diazepino[4′,5′,6′:4,5]phenanthro[2,1,10-def::7,8,9-d′e′f′]diisoquinoline-1,3,9,11 (2H,5H, 9H,11H)-tetraone, and worked up and purified by column chromatography (silica gel, chloroform). Yield 96 mg (54.1%) of the formula (7), m.p. >250° C. R_{f} (silica gel/chloroform): 0.40. UV/Vis (CHCl₃): λₘₐₓ (Eᵣₑₗ)=391 (0.11), 431 (0.14), 460 (0.25), 492 (0.56), 528 (1.00), 582 (0.73). Fluorescence (CHCl₃): λₘₐₓ (Iᵣₑₗ)=594 (1.00), 645 (0.45), 709 nm (0.10). Fluorescence quantum yield (CHCl₃, λ_{exc}=582 nm, E₅₈₂ nm=0.02399 cm⁻¹, reference: (1a) at 1.00): 1.00. Fluorescence quantum yield (CHCl₃, π_{exc}=528 nm, E₅₂₈ nm=0.03272 cm⁻¹, reference: (1a) at 1.00): 0.80. MS (FAB⁺): m/z (%): 1544.4 (0.1) [M⁺-H], 871.5 (0.2) [M⁺-C₄₅H₆₂N₂O₄], 675.4 (0.7) [M⁺-C₅₇H₆₆N₂O₄], 460.3, 391.4, 307.3.

### EXAMPLE 26

2,10-Bis(1-hexylheptyl)-(N-butyl)-6-phenyl[1,3]diazepino[4′,5′,6′:4,5]-phenanthro[2,1,10-def::7,8,9-d′e′f′]diisoquinoline-1,3,9,11(2H,5H,9H,11H)-tetraone. 2,10-Bis(1-hexylheptyl)-6-phenyl[1,3]diazepino[4′,5′,6′:4,5]phenanthro[2,1,10-def::7,8,9-d′e′f′]diisoquinoline-1,3,9,11(2H,5H,9H,11H)-tetraone (100 mg, 0.115 mmol), 1-iodobutane (635 mg, 3.45 mmol) and potassium carbonate (120 mg, 0.868 mmol) were reacted in DMF (5 mL) as for 2,10-bis(1-hexylheptyl)-(N-benzyl)-6-phenyl[1,3]diazepino[4′,5′,6′:4,5]phenanthro[2,1,10-def::7,8,9-d′e′f′]diisoquinoline-1,3,9,11(2H,5H,9H,11H)-tetraone and worked up and purified by column chromatography (silica gel, chloroform). Yield 48 mg (45%) of dark red to black solid of the formula (7), m.p. >250° C. R_{f} (silica gel/chloroform): 0.76. IR (ATR): \tilde{v}=3062.8 w, 2954.3 m, 2922.6 s, 2855.1 s, 1686.5 s, 1644.5 s, 1591.3 m, 1574.4 m, 1529.3 w, 1484.2 w, 1424.0 m, 1408.1 w, 1378.6 w, 1330.3 s, 1252.4 m, 1221.3 w, 1178.6 w, 1101.3 w, 1025.7 w, 874.5 w, 841.9 w, 808.0 m, 772.1 w, 750.0 w, 587.6 w cm⁻¹. ¹H NMR (600 MHz, CDCl₃, 25° C.): δ=0.56 (t, ³J=12.0 Hz, 3H, CH₃), 0.80-0.82 (m, 12H, CH₃), 1.22-1.31 (m, 32H, CH₂), 1.84-1.91 (m, 4H, β-CH), 2.29-2.24 (m, 4H, β-CH), 4.97 (t, ³J=12.0 Hz, 2H, N—CH₂), 5.16-5.24 (m, 2H, N—CH), 7.67-7.68 (m, 3H, CH_{aryl}), 7.99-8.01 (m, 2H, CH_{aryl}), 8.71-8.74 (m, 5H, CH_{perylene}), 10.82 ppm (d, ³J=12.0 Hz, 1H, CH_{perylene}). UV/Vis (CHCl₃): λ_{max (E}ᵣₑₗ)=376.6 (0.79), 395.2 (0.09), 438.8 (0.11), 502.4 (0.16), 539.2 (0.52), 583.8 nm (1.00). Fluorescence (CHCl₃): λₘₐₓ (1)=596.2 (1.00), 647.5 nm (0.45). Fluorescence quantum yield (CHCl₃, λ_{exc}=539 nm, E_{539 nm}=0.01647 cm⁻¹, reference: (1a) at Φ=1.00): 1.00. HRMS (C₆₈H₈₀N₄O₄): calc. m/z=926.5710, found m/z=926.5721, Δ=0.0011.

### EXAMPLE 27

2,10-Bis(1-hexylheptyl)-(N-pentyl)-6-phenyl[1,3]diazepino[4′,5′,6′:4,5]-phenanthro[2,1,10-def::7,8,9-d′e′f′]diisoquinoline-1,3,9,11(2H, 5H, 9H,11H)-tetraone. 2,10-Bis(1-hexylheptyl)-6-phenyl[1,3]diazepino[4′,5′,6′:4,5]phenanthro[2,1,10-def::7,8,9-d′e′f′]diisoquinoline-1,3,9,11(2H,5H,9H,11H)-tetraone (100 mg, 0.115 mmol), 1-iodopentane (683 mg, 3.45 mmol) and potassium carbonate (120 mg, 0.868 mmol) were reacted in DMF (5 mL) as for 2,10-bis(1-hexylheptyl)-(N-benzyl)-6-phenyl[1,3]diazepino[4′,5′,6′:4,5]phenanthro[2,1,10-def::7,8,9-d′e′f′]diisoquinoline-1,3,9,11 (2H, 5H, 9H,11H)-tetraone, and worked up, purified by column chromatography (silica gel, chloroform) to remove unreacted 1-iodopentane, dissolved in toluene and admixed with an excess of DBU, and purified by column chromatography (basic alumina, toluene, the deprotonated starting material is strongly adsorbed). Yield 75 mg (69%) of dark red to black solid of the formula (7).

### EXAMPLE 28

2,10-Bis(1-hexylheptyl)-(N-pentyl)-6-phenyl[1,3]diazepino[4′,5′,6′:4,5]-phenanthro[2,1,10-def::7,8,9-d′e′f′]diisoquinoline-1,3,9,11(2H,5H,9H,11H)-tetraone (variant to Ex. 27). 2,10-Bis(1-hexylheptyl)-6-phenyl[1,3]diazepino[4′,5′,6′:4,5]-phenanthro[2,1,10-def::7,8,9-d′e′f′]diisoquinoline-1,3,9,11 (2H,5H, 9H,11H)-tetraone (100 mg, 0.115 mmol), 1-iodopentane (683 mg, 3.45 mmol) and potassium carbonate (120 mg, 0.868 mmol) were suspended in DMPU (5 mL), heated to 100° C. for 6 h, admixed while still lukewarm with aqueous HCl (2 N, 50 mL, precipitated dye as a red suspension), filtered with suction, dried, taken up in chloroform and purified by column chromatography (silica gel, chloroform) to remove unreacted 1-iodopentane. Yield 90 mg (83%), m.p. >250° C. R_{f} (silica gel/chloroform): 0.59. IR (ATR): \tilde{v}=3061.2 w, 2954.1 m, 2922.7 s, 2854.7 s, 1686.7 s, 1644.5 s, 1591.1 m, 1529.6 w, 1484.3 w, 1454.7 w, 1424.7 w, 1408.1 w, 1378.5 w, 1331.0 s, 1251.8 m, 1222.8 w, 1179.2 w, 1101.9 w, 1025.9 w, 929.9 w, 874.0 w, 842.2 w, 808.4 w, 771.9 w, 749.8 w cm⁻¹. ¹H NMR (600 MHz, CDCl₃, 25° C.): δ=0.57 (t, ³J=7.3 Hz, 3H, CH₃), 0.66-0.75 (br, 2H, CH₂), 0.78-0.83 (m, 12H, CH₃), 0.88-0.96 (m, 2H, CH₂), 1.15-1.43 (m, 32H, CH₂), 1.82-1.95 (m, 4H, β-CH), 2.21-2.40 (m, 4H, β-CH), 4.93 (t, ³J=6.8 Hz, 2H, N—CH₂), 5.15-5.30 (m, 2H, N—CH), 7.66-7.67 (m, 3H, CH_{aryl}), 7.98-7.99 (m, 2H, CH_{aryl}), 8.66-8.73 (m, 5H, CH_{perylene}), 10.77 ppm (d, ³J=6 Hz, 1H, CH_{perylene}). ¹³C NMR (150 MHz, CDCl₃, 25° C.): δ=13.9, 14.3, 22.2, 22.8, 22.9, 27.2, 27.4, 28.4, 28.7, 29.5, 29.6, 32.0, 32.0, 32.7, 49.4, 54.8, 121.5, 122.8, 123.9, 127.0, 127.3, 129.3, 130.2, 130.5, 131.3, 131.6, 135.1, 145.1, 163.9, 164.1, 165.2 ppm. UV/Vis (CHCl₃): λₘₐₓ (Eᵣₑₗ)=377.2 (0.09), 396.0 (0.10), 440.2 (0.11), 502.8 (0.16), 539.8 (0.52), 584.2 nm (1.00). Fluorescence (CHCl₃): λₘₐₓ (Iᵣₑₗ)=596.6 (1.00), 650.1 nm (0.45). Fluorescence quantum yield (CHCl₃, λ_{exc}=540 nm, E₅₄₀ nm=0.01462 cm⁻¹, reference: (1a) at 1.00): 1.00, HRMS (C₆₂H₇₈N₄O₄): calc. m/z=941.5900, found m/z=941.5924, Δ=0.0024. C₆₂H₇₈N₄O₄ (961.3): calc. C, 79.11; H, 5.95; N, 8.14. found C, 78.99; H, 5.91; N, 8.51.

### EXAMPLE 29

2,10-Bis(1-hexylheptyl)-(N-hexyl)-6-phenyl[1,3]diazepino[4′,5′,6′:4,5]-phenanthro[2,1,10-def::7,8,9-d′e′f′]diisoquinoline-1,3,9,11 (2H,5H,9H,11H)-tetraone. 2,10-Bis(1-hexylheptyl)-6-phenyl[1,3]diazepino[4′,5′,6′:4,5]phenanthro[2,1,10-def::-7,8,9-d′e′f′]diisoquinoline-1,3,9,11 (2H, 5H, 9H, 11H)-tetraone (100 mg, 0.115 mmol), 1-bromohexane (570 mg, 3.45 mmol) and potassium carbonate (300 mg, 2.17 mmol) were suspended in DMPU (5 mL), heated to 100° C. for 3 h, admixed while still lukewarm with aqueous HCl (2 N, 50 mL), extracted three times with chloroform (50 mL each time), dried over magnesium sulfate, concentrated by evaporation, taken up in a little chloroform and purified by column chromatography (silica gel, 3:1 chloroform/isohexane) to remove unreacted 1-bromohexane. Yield 86 mg (78%) of the formula (7), m.p. >250° C. R_{f} (silica gel, chloroform): 0.59. ¹H NMR (600 MHz, CDCl₃, 25° C.): δ=0.60 (t, ³J=7.3 Hz, 3H, CH₃), 0.67-0.75 (br, 2H, CH₂), 0.76-0.82 (m, 12H, CH₃), 0.83-0.90 (m, 2H, CH₂), 0.92-1.10 (m, 2 H, CH₂), 1.16-1.44 (m, 34H, CH₂), 1.82-1.96 (m, 4H, β-CH), 2.20-2.40 (m, 4H, β-CH), 4.93 (t, ³J=6.7 Hz, 2H, N—CH₂), 5.15-5.31 (m, 2H, N—CH), 7.61-7.70 (m, 3 H, CH_{aryl}), 7.96-8.01 (m, 2H, CH_{aryl}), 8.60-8.82 (m, 5H, CH_{perylene}), 10.76 ppm (d, ³J=8.04 Hz, 1H, CH_{perylene}). ¹³C NMR (150 MHz, CDCl₃, 25° C.): δ=13.9, 14.3, 22.5, 22.8, 22.9, 25.9, 27.2, 27.4, 28.9, 29.5, 29.6, 31.2, 32.0, 32.1, 32.7, 49.4, 54.8, 121.4, 122.8, 123.8, 127.0, 127.3, 129.3, 130.2, 130.5, 131.3, 131.6, 145.1, 163.9 ppm. UV/Vis (CHCl₃): λₘₐₓ (Eᵣₑₗ)=377.1 (0.11), 394.7 (0.12), 500.9 (0.18), 539.0 (0.53), 583.7 nm (1.00). Fluorescence (CHCl₃): λₘₐₓ (Iᵣₑₗ)=596.8 (1.00), 650.1 nm (0.45). Fluorescence quantum yield (CHCl₃, λ_{exc}=539 nm, E₅₃₉ nm=0.01671 cm⁻¹, reference: (1a) at 1.00): 1.00.

### EXAMPLE 30

2,10-Bis(1-hexylheptyl)-(N-allyl)-6-phenyl[1,3]diazepino[4′,5′,6′:4,5]-phenanthro[2,1,10-def::7,8,9-d′e′f′]diisoquinoline-1,3,9,11(2H, 5H, 9H,11H)-tetraone. 2,10-Bis(1-hexylheptyl)-6-phenyl[1,3]diazepino[4′,5′,6′:4,5]phenanthro[2,1,10-def::-7,8,9-d′e′f′]diisoquinoline-1,3,9,11 (2H,5H, 9H,11H)-tetraone (100 mg, 0.115 mmol), allyl bromide (624 mg, 3.45 mmol) and potassium carbonate (300 mg, 2.17 mmol) were suspended in DMPU (5 mL), heated to 65° C. for 24 h, admixed while still lukewarm with aqueous HCl (2 N, 50 mL), filtered with suction, dried, taken up in a little chloroform and purified by column chromatography (silica gel, 2:1 chloroform/isohexane) to remove unreacted allyl bromide. Yield 87 mg (83%) of the formula (7), m.p. >250° C. R_{f} (silica gel, chloroform): 0.52. ¹H NMR (600 MHz, CDCl₃, 25° C.): δ=0.82 (t, ³J=7.0 Hz, 12H, CH₃), 1.16-1.40 (m, 32H, CH₂), 1.82-1.92 (m, 4H, β-CH), 2.20-2.34 (m, 4H, β-CH), 4.44-4.64 (br, 1H, CH_{allyl}), 4.81-4.88 (br, 1H, CH_{allyl}), 5.15-5.22 (m, 2H, N—CH₂), 5.42-5.60 (br, 3H, N—CH+CH_{allyl}), 7.64-7.68 (m, 3H, CH_{aryl}), 7.95-8.00 (m, 2H, CH_{aryl}), 8.60-8.79 (m, 5H, CH_{perylene}), 10.75 ppm (d, ³J=8.1 Hz, 1H, CH_{perylene}). ¹³C NMR (150 MHz, CDCl₃, 25° C.): δ=14.3, 22.8, 22.8, 27.2, 27.3, 29.5, 29.5, 32.0, 32.1, 32.7, 51.3, 54.9, 55.3, 121.5, 122.8, 123.9, 125.6, 127.0, 127.3, 129.3, 129.9, 130.4, 130.4, 131.5, 132.5, 135.0, 139.6, 144.8, 163.4, 164.1, 165.1 ppm. UV/Vis (CHCl₃): λₘₐₓ (ε)=376.0 (8067), 395.4 (8978), 500.3 (14530), 536.9 (43870), 581.3 nm (82740). Fluorescence (CHCl₃): λₘₐₓ (Iᵣₑₗ)=594.6 (1.00), 644.2 nm (0.32). Fluorescence quantum yield (CHCl₃, λ_{exc}=537.5 nm, E_{537.5} nm=0.01405 cm⁻¹, reference: (1a) at Φ=1.00): 1.00. HRMS (C₆₀H₇₀N₄O₄): calc. m/z=910.5397, found m/z=910.5379, Δ=−0.0018. C₆₀H₇₀N₄O₄ (911.2): calc. C, 79.09; H, 7.74; N, 6.15. found C, 78.87; H, 7.71; N, 5.83.

### EXAMPLE 31

2,10-Bis(1-hexylheptyl)-(N-propargyl)-6-phenyl[1,3]diazepino[4′,5′,6′:-4,5]phenanthro[2,1,10-def::7,8,9-d′e′f′]diisoquinoline-1,3,9,11 (2H,5H, 9H,11H)-tetra-one. 2,10-Bis(1-hexylheptyl)-6-phenyl[1,3]diazepino[4′,5′,6′:4,5]phenanthro[2,1,10-def::7,8,9-d′e′f′]diisoquinoline-1,3,9,11(2H,5H,9H,11H)-tetraone (300 mg, 0.344 mmol), propargyl bromide (1.53 g, 10.3 mmol, 80 percent in toluene) and potassium carbonate (900 mg, 6.517 mmol) were suspended in DMPU (15 mL), heated to 85° C. for 24 h, concentrated by evaporation, admixed with aqueous HCl (2 N, 150 mL), filtered with suction, dried, taken up in a little chloroform and purified by column chromatography (silica gel, 3:1 chloroform/iso-hexane) to remove unreacted allyl bromide. Yield 212 mg (67.7%) of the formula (7), m.p. >250° C. R_{f} (silica gel, chloroform): 0.45. ¹H NMR (600 MHz, CDCl₃, 25° C.): δ=0.82 (t, ³J=6.8 Hz, 12H, CH₃), 1.15-1.44 (m, 32H, CH₂), 1.82-1.95 (m, 4H, β-CH), 1.99 (s, 1H, C≡CH), 2.21-2.36 (m, 4H, β-CH), 5.14-5.30 (m, 2H, N—CH₂), 5.67 (s, 2H, CH_{propargyl}), 7.65-7.75 (m, 3H, CH_{aryl}), 7.99-8.03 (m, 2H, CH_{aryl}), 8.53-8.78 (m, 5 H, CH_{perylene}), 10.68 ppm (d, ³J=8.0 Hz, 1H, CH_{perylene}). ¹³C NMR (150 MHz, CDCl₃, 25° C.): δ=14.3, 14.3, 22.7, 22.8, 22.9, 27.2, 27.4, 29.5, 29.6, 29.9, 32.0, 32.1, 32.7, 32.8, 39.7, 54.9, 55.4, 121.6, 122.5, 122.8, 123.1, 124.0, 125.6, 126.9, 127.3, 129.1, 129.3, 129.5, 130.5, 131.4, 131.8, 132.6, 134.9, 139.1, 144.7, 162.9, 164.0, 165.1 ppm. UV/Vis (CHCl₃): λₘₐₓ (Eᵣₑₗ)=375.2 (0.10), 395.3 (0.11), 434.6 (0.11), 498.4 (0.18), 534.8 (0.54), 579.0 nm (1.00). Fluorescence (CHCl₃): λₘₐₓ (Iᵣₑₗ)=591.6 (1.00), 643.0 nm (0.47). Fluorescence quantum yield (CHCl₃, λ_{exc}=543 nm, E_{537.5 nm}=0.008991 cm⁻¹, reference: (1a) at 1.00): 0.17. HRMS (C₆₀H₆₈N₄O₄): calc. m/z 908.5241, found m/z 908.5216, Δ=−0.0025. C₆₀H₆₈N₄O₄ (909.2): calc. C, 79.26; H, 7.54; N, 6.16. found C, 79.13; H, 7.62; N, 6.14.

### EXAMPLE 32

2,9-Bis(1-hexylheptyl)-bis-[1,3]diazepino[4′,5′,6′:1,12;4″,5″, 6″:6,7]-perylo[3,4-cd:9,10-c′d′]dipyridine-1,3,9,11 (2H, 5H,10H,13H)-tetraone. 2,9-Bis(1-hexylheptyl)-bis-[1,3]diazepino[4′,5′,′:1,12;4″,5″,6″:6,7]perylo[3,4-cd:9,10-c′d′]dipyridine-1,3,9,11(2H,5H,10H,13H)-tetraone according to Example 22 (90 mg, 0.091 mmol), p-tert-butylbenzyl bromide (800 mg, 3.52 mmol) and potassium carbonate (600 mg, 4.34 mmol) were suspended in 10 mL of DMPU, heated to 100° C. for 6 h, admixed while still warm with aqueous HCl solution (2 N, 50 mL), extracted by shaking three times with chloroform, dried over magnesium sulfate, concentrated by evaporation, and purified by column chromatography (silica gel, 3:1 chloroform/isohexane). Yield 72 mg (62%) of black solid of the formulae (9) and (10), m.p. >250° C. R_{f} (silica gel, 3:1 chloroform/isohexane)=0.62. ¹H NMR (600 MHz, CDCl₃, 25° C., cis product): δ=0.82 (q, ³J=7.04 Hz, 12H, CH₃), 1.05 (s, 18H, CH_{3,tert-butyl}), 1.17-1.40 (m, 32H, CH₂), 1.82-1.91 (m, 2H, CH₂), 1.97-2.12 (m, 4H, CH₂), 2.23-2.33 (m, 2H, CH₂), 5.10-5.26 (m, 2H, CH—N), 6.12 (s, 6H, cis-N—CH₂), 6.44 (d, ³J=7.7 Hz, 2H, cis-CH_{aromatic}), 6.90-6.96 (br, 2H, CHₐᵣₒₘₐₜ), 7.57-7.67 (br, 6H, CHₐᵣₒₘₐₜ), 7.94-8.07 (br, 4H, CHₐᵣₒₘₐₜ), 8.73-8.83 (br, 2H, CH_{perylene}), 10.84 ppm (d, ³J=7.64 Hz, 2H, CH_{perylene}). ¹H NMR (600 MHz, CDCl₃, 25° C., trans product): δ=0.82 (q, ³J=7.04 Hz, 12H, CH₃), 1.05 (s, 18H, CH_{3,tert-butyl}), 1.17-1.40 (m, 32H, CH₂), 1.82-1.91 (m, 2H, CH₂), 1.97-2.12 (m, 4H, CH₂), 2.23-2.33 (m, 2H, CH₂), 5.10-5.26 (m, 2H, CH—N), 6.06 (s, 6H, trans-N—CH₂), 6.37 (d, ³J=7.7 Hz, 2H, trans-CH_{aromatic}), 6.90-6.96 (br, 2H, CHₐᵣₒₘₐₜ), 7.57-7.67 (br, 6H, CHₐᵣₒₘₐₜ), 7.94-8.07 (br, 4H, CHₐᵣₒₘₐₜ), 8.73-8.83 (br, 2H, CH_{perylene}), 10.84 ppm (d, ³J=7.64 Hz, 2H, CH_{perylene}). ¹³C NMR (150 MHz, CDCl₃, 25° C.): δ=14.3, 14.3, 22.8, 23.0, 27.2, 27.8, 29.5, 29.7, 31.3, 31.3, 32.0, 32.1, 32.7, 33.0, 34.5, 51.7, 51.8, 54.7, 55.8, 76.9, 108.4, 108.8, 121.9, 125.3, 125.6, 125.8, 126.1, 126.3, 127.5, 128.8, 129.3, 130.2, 130.3, 130.5, 131.2, 131.4, 131.9, 133.9, 134.0, 135.1, 139.7, 140.0, 143.7, 150.4, 150.6, 163.1, 163.7, 163.8, 164.1, 164.4 ppm. UV/Vis (CHCl₃): λₘₐₓ (Eᵣₑₗ)=383.6 (0.11), 404.2 (0.13), 465.4 (0.18), 488.8 (0.19), 535.0 (0.11), 577.4 (0.41), 629.8 (1.00). Fluorescence (CHCl₃): λₘₐₓ (Iᵣₑₗ)=642.3 (1.00), 701.9 nm (0.34). Fluorescence quantum yield (CHCl₃, λ_{exc}=577 nm, E_{577 nm}=0.014317 cm⁻¹, reference: (1a) at 1.00): 1.00. MS (DEI⁺, 70 eV): m/z (%)=1281.3 (16.4), 1280.3 (48.7), 1279.3 (96.0), 1278.3 (100.0) [M⁺], 1134.3 (3.3), 1133.3 (8.4), 1132.3 (11.5) [M⁺-p-tert-butylbenzyl], 1098.2 (2.7), 1097.2 (4.6), 1096.2 (4.2) [M⁺-C₁₃H₂₆]. C₈₆H₉₈N₆O₄ (1279.7): calc. C, 80.71; H, 7.72; N, 6.57. found C, 80.35; H, 7.84; N, 6.46.

1. A compound of the formula 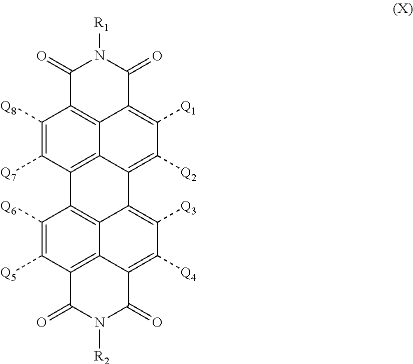
in which one pair Q₁ and Q₂ or one pair Q₂ and Q₃, and optionally from 0 to 3 pairs Q₃ and Q₄, Q₅ and Q₆, Q₆ and Q₇ and/or Q₇ and Q₈, in each case in pairs, together form a heterocyclic ring 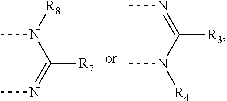
the remaining Q₁, Q₄, Q₅ and Q₈ which do not form a heterocyclic ring are each hydrogen, and the remaining Q₃, Q₆ and Q₇ which do not form a heterocyclic ring are each independently R₅ or R₆, preferably 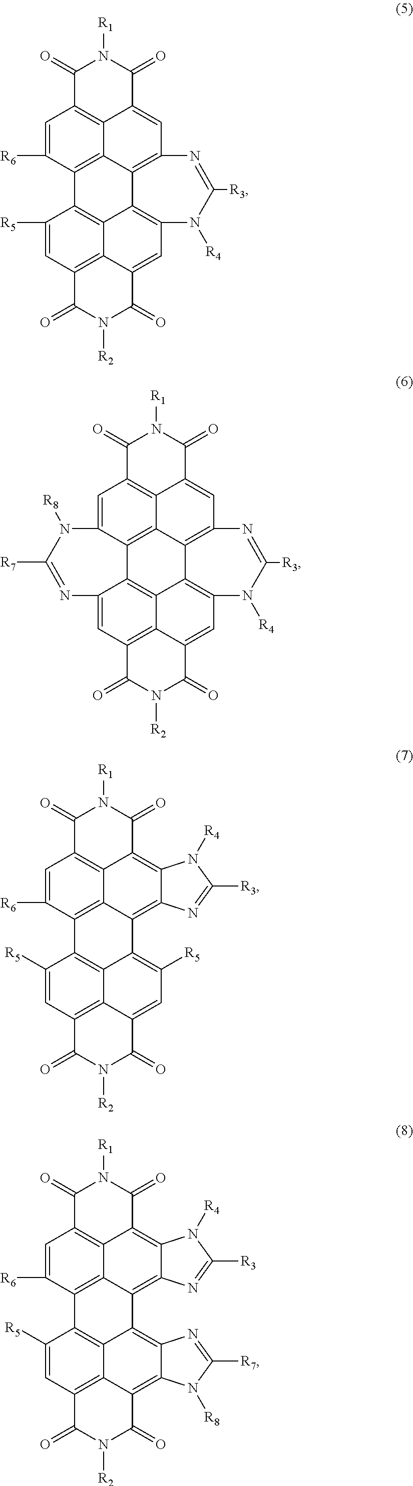 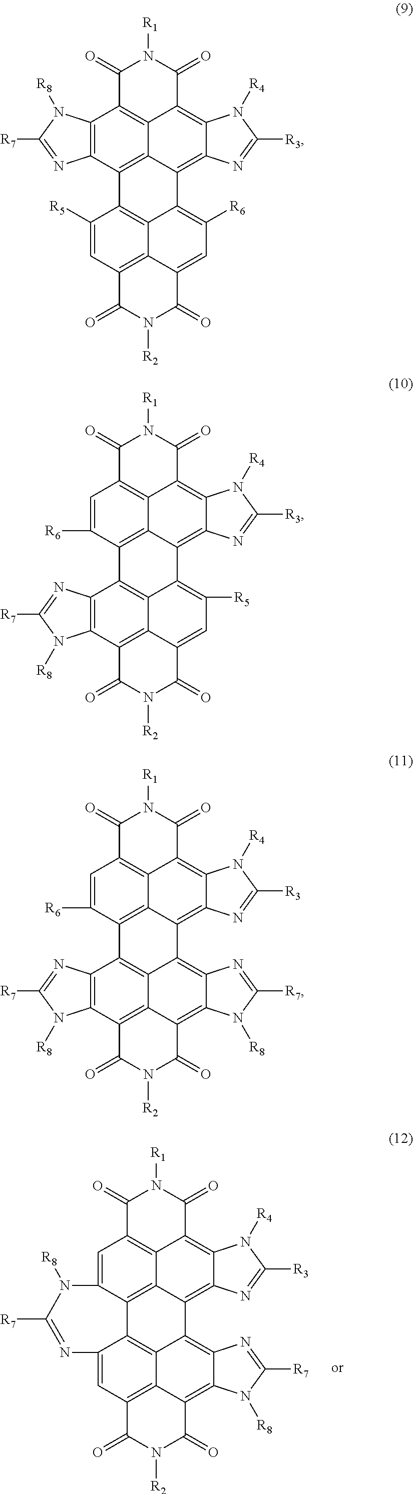 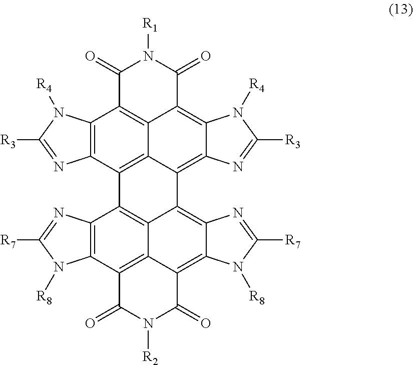
in which
R₁ to R₈ are each independently H, F, Cl, Br, I or {linear C₁-C₃₇alkyl}(-R₉)ₘ, m is from 0 to 12,
R₉, and in the case of multiple R₉ each R₉ independently of all other R₉, is H, F, Cl, Br, I, CN or {linear C₁-C₁₈alkyl}(-R₁₀)ₙ, n is from 0 to 6,
R₁₀, and in the case of multiple R₁₀ each R₁₀ independently of all other R₁₀, is H, F, Cl, Br, I, CN or {linear C₁-C₁₈alkyl},
where no or from 1 to 10 —CH₂— units in each {linear C₁-C₃₇alkyl}, if appropriate independently of all other {linear C₁-C₃₇alkyl}, may be replaced by R₁₁, and/or no or from 1 to 6 —CH₂— units in {linear C₁-C₁₈alkyl}, and in the case of multiple {linear C₁-C₁₈alkyl} in each {linear C₁-C₁₈alkyl} independently of all other {linear C₁-C₁₈alkyl}, may be replaced by R₁₂, and
R₁₁ and R₁₂ are each independently, and in the case of multiple R₁₁ and R₁₂ each R₁₁ or R₁₂ independently of all other R₁₁ and R₁₂, is —CO—, —O—, —S—, —Se—, —Te—, cis or trans —CH═CH—, cis or trans —N═CH—, —C≡C—, 1,2-, 1,3- or 1,4-phenylene, 2,3-, 2,4-, 2,5-, 2,6-, 3,4- or 3,5-pyridinediyl, 2,3-, 2,4-, 2,5- or 3,4-thiophenediyl, 1,2-, 1,3-, 1,4-, 1,5-, 1,6-, 1,7-, 1,8-, 2,3-, 2,6- or 2,7-naphthylene, in which 0, 1 or 2 ═CH— may be replaced by ═N—, or 1,2-, 1,3-, 1,4-, 1,5-, 1,6-, 1,7-, 1,8-, 1,9-, 1,10-, 2,3-, 2,6-, 2,7-, 2,9-, 2,10- or 9,10-disubstituted anthracylene in which 0, 1 or 2 ═CH— may be replaced by ═N—,
or any two R₉, R₁₀, R₁₁ and R₁₂ substituents together form, in a single pair or multiple pairs, a direct bond, so as to form rings, preferably cyclohexane or benzene rings,
where R₄ may optionally be bonded by a direct bond to R₄ of a second molecule, and/or R₈ may be bonded by a direct bond to R₈ of a second molecule, so as to form dimers, trimers, tetramers or higher oligomers.

2. A compound according to claim 1, in which {linear C₁-C₃₇alkyl}(-R₉)ₘ is an unsubstituted or substituted hydrocarbon radical which comprises from 1 to 60, preferably from 1 to 37 and more preferably from 1 to 18 carbon atoms, where this hydrocarbon radical in the case of R₃ and/or R₈ preferably forms a phenyl radical by trisubstitution of —CH₂— with —CH═CH— and a direct bond between 2 R₉ substituents.

3. A process for preparing a compound according to claim 1 or 2, which comprises reacting a perylenetetracarboximide with an aromatic nitrile in the presence of a strong base, appropriately in the presence of oxygen.

4. The process according to claim 3, in which the aryl nitrile, based on the perylenetetracarboximide, is used in an equimolecular amount or in excess, and the reaction temperature is from 80 to 300° C., preferably from 100 to 200° C., especially approximately 160° C.

5. A process for preparing a compound according to claim 1 or 2, which comprises reacting a perylenetetracarboximide of the formula (X′) in which, as the sole difference from the formula (X), Q₄, Q₄ and Q₆, or Q₄ and Q₇ are each arylamido groups in which aryl is selected from R₃ or R₈ groups, in the presence of a strong base, appropriately with sodium amide.

6. The process according to claim 3, 4 or 5, in which the reaction takes place in the presence of an inert solvent.

7. A process for preparing a compound according to claim 1 or 2, wherein a compound according to claim 1 or 2 in which R₄, R₈ or R₄ and R₈ are each H is alkylated, preferably methylated, at least one R₄ or R₈, preferably in a dipolar aprotic solvent.

8. A composition comprising a compound according to claim 1 or 2 and a weak base, preferably a primary, secondary or tertiary amine.

9. A process for increasing the Stokes shift of a compound according to claim 1 or 2, preferably by the ESPT mechanism, which comprises adding a weak base, preferably a primary, secondary or tertiary amine, to the compound according to claim 1 or 2.

10. The use of a compound according to claim 1 or 2 as a colorant, preferably as a pigment, more preferably in distempers and related colors, paper inks, printing inks, solventborne and waterborne inks, paints or coating materials, as rheology improvers, as fluorescent colorants, in optical recording materials, in OLEDs (organic light-emitting diodes), in photovoltaic cells, for bulk coloring of polymers, for coloring of natural substances, as a mordant dye, in electrophotographic systems, for security and identification markings, as a component of colorant compositions, in nonlinear optical elements, in lasers, for frequency conversion of light, in display elements, as a starting material for superconductive organic materials, for solid fluorescent markings, for decorative or artistic purposes, or as a tracer in biochemistry, medicine, technology or natural science.

11. The use according to claim 10, in which the compound according to claim 1 or 2 is used in a fluorescent solar collector, in a display, in a cold light source, in a chemiluminescent glow stick, in an integrated semiconductor circuit, in a luminescence detection method or in a photoconductor.

12. A process for inducing fluorescence in the range from 500 to 1000 nm, which comprises irradiating a compound according to claim 1 or 2 with electromagnetic radiation of wavelength from 250 to 600 nm, preferably visible light of wavelength from 400 to 600 nm.

13. The process according to claim 12, in which the fluorescence is used to generate power or heat, or to conduct a chemical reaction.

14. A process for detecting fluorescence in the range from 500 to 1000 nm, which comprises inducing the fluorescence by irradiating a compound according to claim 1 or 2 with electromagnetic radiation of wavelength from 250 to 600 nm, preferably visible light of wavelength from 400 to 600 nm.

15. The process according to claim 14, in which the detected fluorescence is collected and converted to an analog or digital signal or to energy.
